(19) United States
(12) Patent Application Publication
(10) Pub. No.: US 2018/0078239 A1
(43) Pub. Date: Mar. 22, 2018

(54) SYSTEMS AND METHODS FOR DISPLAYING MEDICAL IMAGES

(71) Applicant: Evena Medical, Inc., Los Altos, CA (US)

(72) Inventors: Frank J. Ball, Roseville, CA (US); Ignacio E. Cespedes, Folsom, CA (US); Melvyn L. Harris, Folsom, CA (US); David J. Gruebele, Folsom, CA (US); Paul M. Hoseit, El Dorado Hills, CA (US)

(21) Appl. No.: 15/581,811

(22) Filed: Apr. 28, 2017

Related U.S. Application Data
(63) Continuation of application No. 14/522,188, filed on Oct. 23, 2014, now abandoned.
(60) Provisional application No. 61/895,302, filed on Oct. 24, 2013.
(60) Provisional application No. 61/988,098, filed on May 2, 2014.

(51) Int. Cl. A61B 8/00; A61B 5/00; A61B 8/08

(52) U.S. Cl. CPC A61B 8/462; A61B 5/7445; A61B 5/0035; A61B 5/0077; A61B 8/5261; A61B 8/4416; A61B 8/565; A61B 8/5207; A61B 8/464

(57) ABSTRACT

Various embodiments relate to methods and systems for displaying an ultrasound image of a patient's target body portion using a wearable display module. A wearable display module can be mounted on a user's head. A wearable display module can display ultrasound images as a heads up display so that a user can view an ultrasound image while maintaining lines of sight with other viewpoints. Various embodiments relate to methods and systems for displaying both an ultrasound image and a light image (e.g., a near infrared image). A single display can be configured to display the light image and the ultrasound image on a single surface, in close proximity, and at the same time or in sequence. This disclosure provides in some embodiments for the use of wireless communication between various imaging probes, sensors, image processors, and display modules in order to facilitate more effective viewing of images.

### RELATED APPLICATIONS

This application is a continuation of U.S. patent application Ser. No. 14/522,188, filed Oct. 23, 2014, and titled SYSTEMS AND METHODS FOR DISPLAYING MEDICAL IMAGES, which claims the benefit under 35 U.S.C. §119(e) of U.S. Provisional Patent Application No. 61/895,302, filed Oct. 24, 2013, and titled SYSTEMS AND METHODS FOR DISPLAYING MEDICAL IMAGES, and U.S. Provisional Patent Application No. 61/988,098, filed May 2, 2014, and titled SYSTEMS AND METHODS FOR DISPLAYING MEDICAL IMAGES. The entire contents of each of the above-identified applications are hereby incorporated by reference herein and made a part of this specification.

### INCORPORATION BY REFERENCE

U.S. patent application Ser. No. 13/802,604 (the '604 Application), titled VEIN IMAGING SYSTEMS AND METHODS, filed on Mar. 13, 2013, and published as U.S. Patent Publication No. US 2014/0039309 on Feb. 6, 2014, is hereby incorporated by reference in its entirety.

### BACKGROUND

### Field of the Disclosure

Some embodiments of this disclosure relate to systems and methods for displaying medical images, such as subcutaneous images taken of a patient with medical imaging technology.

### Description of the Related Art

Certain imaging technologies such as near infrared (NIR) light imaging and ultrasound imaging can allow a doctor or healthcare professional to examine subcutaneous tissue of a patient. Access to such images can be useful for effective treatment and/or diagnosis, particularly in emergency response settings. However, many methods and systems for displaying medical images require consulting stationary screens adapted only for displaying a single type of image. Such limitations can cause delay in treatment and/or diagnosis.

### SUMMARY OF SOME EMBODIMENTS

A method for displaying an ultrasound image can include identifying a target body portion of a patient to be imaged; emitting ultrasound signals from an ultrasound probe, wherein the ultrasound signals can cause echo signals to be reflected from tissue within the target body portion; receiving the echo signals; generating an ultrasound image of the target body portion based at least in part on the echo signals; and displaying the ultrasound image on a head-mounted display.

The method can include transmitting the ultrasound image to a remote display accessible to a remote healthcare professional; receiving treatment instructions from the remote healthcare professional; and administering treatment based at least in part on the instructions from the remote healthcare professional.

The method can include generating echo signal data based at least in part on the echo signals; and transmitting the echo signal data wirelessly to an image processor that generates the ultrasound image.

The method can include transmitting the ultrasound image wirelessly to the head-mounted display.

The method can include attaching the head-mounted display to a user. The attaching can be achieved by the user wearing a helmet that supports the head-mounted display such that the ultrasound image displayed on the head-mounted display is configured to be visible to the user wearing the helmet. The attaching can be achieved by the user wearing an eyewear frame that supports the head-mounted display such that the ultrasound image displayed on the head-mounted display is configured to be visible to the user wearing the eyewear frame. The attaching can be achieved by the user wearing a headband that supports the head-mounted display such that the ultrasound image displayed on the head-mounted display is configured to be visible to the user wearing the headband.

A system for displaying an ultrasound image can include an ultrasound probe configured to emit ultrasound signals and detect echo signals reflected from tissue in a target body portion of a patient; an image processor configured to receive echo signal data that is based at least in part on the echo signals received by the ultrasound probe and generate an ultrasound image of the target body portion based at least in part on the echo signal data; and a wearable display configured to receive the ultrasound image of the target body portion from the image processor and display the ultrasound image.

The system can include a remote display accessible to a remote healthcare professional and configured to receive and display the image of the target body portion.

The system can include a communications module that is configured to deliver the ultrasound image wirelessly to a remote display accessible to a remote healthcare professional.

The system can include a communications module that is configured to deliver the ultrasound image wirelessly to the wearable display module.

The wearable display can be configured to display the image of the target body portion on a heads-up display.

The wearable display can include an at least partially transparent surface.

The system can include a helmet that supports the wearable display such that the ultrasound image displayed by the wearable display is configured to be visible to a person wearing the helmet. The system can include an eyewear frame that supports the wearable display such that the ultrasound image displayed by the wearable display is configured to be visible to a person wearing the eyewear frame. The system can include a headband that supports the wearable display such that the ultrasound image displayed by the wearable display is configured to be visible to a person wearing the headband.

An image display system can include an ultrasound probe; a processor configured to generate an image based at least in part on data from the ultrasound probe; and a wearable display configured to display the image.

The wearable display can be configured to display the image as a heads-up display image.

The processor can be capable of wireless communication with at least one of the ultrasound probe and/or the wearable display.

The system can include a remote display configured to display the image.

The system can include a communication interface configured to wirelessly transmit the ultrasound image to a remote display.

The wearable display module can be a head-mounted display configured to display the image to the wearer regardless of the orientation of the wearer's head.

A method for displaying medical images of a target body portion of a patient can include identifying a target body portion to be imaged; emitting ultrasound signals from an ultrasound probe that cause echo signals to be reflected from the target body portion; providing echo signal data based at least in part on the reflected echo signals; generating an ultrasound image of the target body portion from the echo signal data that provides a subcutaneous cross-sectional view of the target body portion; displaying the ultrasound image on a display; illuminating the target body portion with near infrared light; receiving near infrared light from the target body portion onto a light sensor; generating a near infrared image of the target body portion based at least in part on the near infrared light received by the light sensor; and displaying the near infrared image on the same display that displays the ultrasound image.

The ultrasound image and the near infrared light image can be displayed simultaneously.

The ultrasound image and the near infrared image can be displayed at different times on the same display.

The method can include displaying one of the ultrasound image and the near infrared image on the display at a first time; and in response to user input received by a user interface, displaying the other of the near infrared image and the ultrasound image on the display at a second time.

The display can be a head-mounted display. The display can include a heads-up display.

Illuminating the target body portion with near infrared light can include illuminating the target body portion with multiple lines of near infrared light emitted from multiple near infrared light sources. Illuminating the target body portion with near infrared light can include illuminating the target body portion with different lines of near infrared light at different times.

A system for displaying medical images of a target body portion of a patient can include an ultrasound probe configured to emit ultrasound signals and receive echo signals reflected from a target body portion of a patient; an ultrasound image processor configured to generate an ultrasound image of the target body portion based at least in part on the echo signals received by the ultrasound probe; a near infrared light source configured to direct near infrared light onto the target body portion; a light sensor configured to receive near infrared light reflected from the target body portion; a near infrared light image processor configured to generate a near infrared image of the target body portion based at least in part on the near infrared light received by the light sensor; and a display configured to receive both the ultrasound image and the near infrared image and display both the ultrasound image and the near infrared image.

The ultrasound image processor and the near infrared processor are implemented using a single computer processor.

The display can be configured to display both the ultrasound image and the near infrared image simultaneously.

The system can include a user interface configured to receive input from a user to select the ultrasound image or the near infrared image, and the display can be configured to display the ultrasound image in response to a user selection of the ultrasound image, and the display can be configured to display the near infrared image in response to a user selection of the near infrared image.

The system can include a remote display configured to be accessible to a remote healthcare professional and configured to receive both the ultrasound image and the near infrared image and display the two images.

The display can be configured to display both the ultrasound image and the near infrared image as heads-up display images.

The display can be capable of being physically mounted on a user.

The near infrared light source can include multiple light emitters configured to emit different lines of near infrared light. The system can be configured to illuminate the target body portion with light from each of the multiple light emitters simultaneously. The system can include a user interface that includes one or more user input elements, wherein the multiple light emitters are configured to change the relative intensity of the emitted lines of near infrared light in response to input received by the one or more user input elements. The system can be configured to pulse the multiple light emitters to illuminate the target body portion with different wavelengths of near infrared light at different times.

A system for displaying medical images of a target area can include an ultrasound probe configured to emit ultrasound signals into a target area and receive echo signals from the target area; a light emitter configured to direct light onto the target area; a light sensor configured to receive light from the target area; a controller configured to produce an ultrasound image based at least in part on the echo signals and configured to produce a light image based at least in part on the light received by the light sensor; and a display configured to display both the ultrasound image and the light image.

The light emitter can be configured to emit near infrared (NIR) light.

The display can be configured to display both the ultrasound image and the light image simultaneously.

The display can be capable of being physically mounted on a user.

The display can be configured to display both the ultrasound image and the light image as heads-up display images.

The light emitter can include at least first and second light emitters each having different near infrared emission spectra. The system can be configured to illuminate the target area with light from each of the multiple light emitters simultaneously.

The system can include a user interface that includes one or more user input elements, wherein the multiple light emitters are configured to change the relative intensity of the emitted light from the respective first and second light emitters in response to input received by the one or more user input elements.

The system can be configured to pulse the multiple light emitters to illuminate the target area with different wavelengths of near infrared light at different times.

A method of displaying an ultrasound image can include emitting ultrasound signals from an ultrasound probe, wherein the ultrasound signals cause echo signals to be reflected from tissue within a target body portion of a patient; receiving the echo signals reflected from the tissue within the target body portion; generating an ultrasound image of the target body portion based at least in part on the echo signals; determining a position of the ultrasound probe relative to a head-mounted display; and displaying the ultrasound image on the head-mounted display at a location that is based at least in part on the position of the ultrasound probe relative to the head-mounted display, wherein the location of the ultrasound image on the head-mounted display is configured such that the ultrasound image is overlaid over the target body portion.

The method can include determining that the position of the ultrasound probe relative to the head-mounted display has changed; and moving the location of the ultrasound image on the head-mounted display such that the ultrasound image remains overlaid over the target body portion.

Determining the position of the head-mounted display relative to the ultrasound probe can include receiving light onto a light sensor and analyzing image data that is based on the light received by the light sensor.

The light sensor can be coupled to the head-mounted display, and the ultrasound probe can include one or more position identifiers, and analyzing the image data can include identifying the one or more position identifiers in the image data.

A system for displaying an ultrasound image can include an ultrasound probe configured to emit ultrasound signals and to receive echo signals; a display; and a controller configured to generate an ultrasound image based at least in part on the echo signals, wherein the controller is configured to display the ultrasound image on the display at a location that is based at least in part on a position of the ultrasound probe.

The display can be configured to be coupled to a user. The system can include a head-mounted unit that supports the display.

The controller can be configured to display the ultrasound image at the location based at least in part on a position of the ultrasound probe relative to the display.

The controller can be configured to display the ultrasound image at the location based at least in part on a position of an operator.

The controller can be configured to display the ultrasound image at the location such that the ultrasound image is overlaid over a target body portion being imaged.

The controller can be configured to move the location of the ultrasound image on the display in response to a change in the position of the ultrasound probe. The controller can be configured to move the location of the ultrasound image on the display in response to a change in the position of the ultrasound probe relative to the display. The controller can be configured to move the location of the ultrasound image on the display in response to a change in the position of the ultrasound probe relative to an operator.

The display can include a transparent stationary display configured to be positioned between an operator and the target body portion.

Various display disclosed herein can be configured to be mounted on an articulated arm slidably attached to a vertical support.

A system for displaying medical images of a target body portion of a patient can include a near infrared light source configured to direct near infrared light onto the target body portion, the near infrared light source comprising multiple light emitters having different respective emission spectra; a user interface configured to responsively adjust the relative intensities of the light emitters based on user input; at least one light sensor configured to receive near infrared light from the target body portion; a near infrared light image processor configured to generate a near infrared image of the target body portion based at least in part on the near infrared light received by the light sensor; and a display configured to display the near infrared image.

The near infrared light source can include a first light emitter configured to emit light having a peak in the wavelength range of about 700 nm to about 800 nm; a second light emitter configured to emit light having a peak in the wavelength range of about 800 nm to about 900 nm; and a third light emitter configured to emit light having a peak in the wavelength range of about 900 nm to about 1100 nm.

Various near infrared light sources disclosed herein can include multiple light emitters configured to emit different lines of near infrared light, and the systems disclosed herein can include a user interface configured to responsively adjust the relative intensities of the light emitters based on user input received by the user interface.

Various light emitters disclosed herein can include at least first and second light emitters having different near infrared spectral outputs, and the systems disclosed herein can include a user interface configured to responsively adjust the relative intensities of the first and second light emitters based on user input received by the user interface.

A medical imaging system can include a display; a housing supporting the display; a near infrared light source configured to emit near infrared light onto a target area; a light sensor configured to receive near infrared light from the target area; one or more input modules configured to receive input from one or more of an ultrasound probe, a spectrometer, an electrocardiogram (EKG) device, an X-ray device; a magnetic resonance imaging (MM) device, a pulse oximeter, a blood pressure monitor, a digital stethoscope, a thermometer, an otoscope, and an examination camera; and a controller comprising one or more hardware processors inside the housing, the controller configured to produce an image for the display based at least in part on the near infrared light received by the light sensor, and wherein the controller is configured to produce an image and/or data for the display based at least in part on input received from the one or more input modules.

The near infrared light source can include multiple light emitters configured to emit light having different near infrared spectra.

The system can be configured to illuminate the target area with light from each of the multiple light emitters simultaneously.

The system can include a user interface that includes one or more user input elements, and the multiple light emitters can be configured to change the intensity of the emitted light in response to input received by the one or more user input elements.

The system can be configured to pulse the multiple light emitters to illuminate the target area with different wavelengths of near infrared light at different times.

The system can include a user interface, and the controller can be responsive to user input received via the user interface to toggle the display between a near infrared image and an image that is based on input received from the one or more input modules.

The input module can include a wireless input module configured to receive a wireless signal from one or more of the ultrasound probe, the spectrometer, the electrocardiogram (EKG) device, the X-ray device; the magnetic resonance imaging (MRI) device, the pulse oximeter, the blood pressure monitor, the digital stethoscope, the thermometer, the otoscope, and the examination camera

The input module can include an electrical port for a wired connection.

The controller can be configured to display a near infrared image and an image that is based on input received from the one or more input modules simultaneously.

The system of can include an ultrasound probe coupled to the one or more input modules.

A system for displaying medical images of a target area can include an ultrasound probe configured to emit ultrasound signals into a target area and receive echo signals from the target area; a light emitter configured to direct light onto the target area; a light sensor configured to receive light from the target area; a set of electrodes configured to detect electrical activity; a controller configured to produce an ultrasound image based at least in part on the echo signals, a light image based at least in part on the light received by the light sensor, and electrocardiogram information based at least in part on the electrical activity detected by the electrodes; and a display configured to display the ultrasound image, the light image, and the electrocardiogram information.

The set of electrodes can be configured to detect electrical activity indicative that the tip of a peripheral inserted central catheter has reached a location within a patient.

A system for displaying medical images of a target area can include an ultrasound probe configured to emit ultrasound signals into a target area and receive echo signals from the target area; a light emitter configured to direct light onto the target area; a light sensor configured to receive light from the target area; a spectrometer configured to receive light from the target area; a controller configured to produce an ultrasound image based at least in part on the echo signals, a light image based at least in part on the light received by the light sensor, and spectroscopic information based at least in part on the light detected by the spectrometer; and a display configured to display the ultrasound image, the light image, and the spectroscopic information.

Various systems disclosed herein can include a thermometer configured to detect a temperature associated with the target area, wherein in the controller is further configured to produce a temperature reading based at least in part on the temperature detected by the thermometer and wherein the display is further configured to display the temperature reading.

Various displays disclosed herein can be configured to display the ultrasound image and the near infrared light image at least partially overlaid one on top of the other.

Various displays disclosed herein can be configured to display the ultrasound image and the light image at least partially overlaid one on top of the other.

Various systems disclosed herein can include a visible light source configured to emit visible light onto the target body portion.

Various systems disclosed herein can include a visible light source configured to emit visible light onto the target area, and wherein the light emitter is configured to emit near infrared light.

A medical imaging system can include a near infrared light source configured to emit near infrared light onto a target area; a light sensor configured to receive light from the target area; a display; a controller configured to produce an image for the display based on the near infrared light received by the light sensor; and a visible light source configured to emit visible light onto the target area.

The visible light source can be a red light source. The visible light source is an orange light source.

A method of recording attempts to locate a vein can include emitting near infrared light on a target area; detecting near infrared light received from the target area; displaying a near infrared image based on the near infrared light received from the target area; receiving an input indicating that a vein with a satisfactory venous access site was not identified in the near infrared image; storing an indication of an attempt to identify a vein with a suitable venous access site using near infrared imaging; emitting an ultrasound signal into the target area; receiving echo signals reflected from the target area; and displaying an ultrasound image based on the echo signals.

The indication of the attempt can include a near infrared image based at least in part on the near infrared light received from the target area.

The indication of the attempt can include a patient identifier.

A system for performing and recording attempts to locate a vein can include a near infrared light emitter configured to emit near infrared light onto a target area; a near infrared light detector configured to detect near infrared light received from the target area; an ultrasound probe configured to emit an ultrasound signal into the target area and receive echo signals from the target area; and a controller comprising one or more hardware processors, the controller configured to store an indication of an attempt to identify a vein with a suitable venous access site using near infrared imaging.

The indication of the attempt can include a near infrared image based at least in part on the near infrared light received from the target area.

The indication of the attempt can include a patient identifier.

The system can include a display configured to display both a near infrared image based on the light received by the near infrared light detector and an ultrasound image based on the echo signals received by the ultrasound probe.

A method of accessing a patient's vasculature can include illuminating a target area with near infrared light; positioning a light sensor to receive near infrared light from the target area; viewing a near infrared image on a display, the near infrared image based on the near infrared light received by the light sensor; emitting an ultrasound signal into the target area using an ultrasound probe; receiving echo signals reflected from the target area using an ultrasound probe; viewing an ultrasound image on the same display used for viewing the near infrared image, the ultrasound image based on the echo signals received by the ultrasound probe; identifying or confirming a vein in the target area using the ultrasound image; and inserting a medical implement into the vein.

The method can include using the near infrared image and/or the ultrasound image to facilitate the inserting of the medical implement into the vein.

The method can include identifying a vein using the near infrared image; and confirming the vein using the ultrasound image.

The method can include illuminating the target area with near infrared light to produce a near infrared image on the display after confirming the vein using the ultrasound image; and using the near infrared image to facilitate the inserting of the medical implement into the vein.

The method can include using the ultrasound image to facilitate the inserting of the medical implement into the vein.

A method for assessing the usefulness of near infrared imaging for identifying a vein having a suitable venous access site on a patient can include accessing patient information comprising one or more of the patient' weight, Body Mass Index (BMI), blood pressure, temperature, skin color, past medical history, arm dominance, and kidney function; determining, using one or more computer processors, a score indicative of the likelihood that near infrared imaging would be useful for identifying a vein having a suitable venous access site, the determination based at least in part on the patient information; and outputting the score.

Accessing patient information can include accessing a hospital information system (HIS) and reading the patient information from the hospital information system (HIS).

Accessing patient information can include performing one or more measurements with one or more medical instruments to produce at least some of the patient information.

A system for assessing the usefulness of near infrared imaging for identifying a vein having a suitable venous access site on a patient can include computer-readable storage configured to store patient information comprising one or more of the patient' weight, Body Mass Index (BMI), blood pressure, temperature, skin color, past medical history, arm dominance, and kidney function; at least one processor configured to determine a score indicative of the likelihood that near infrared imaging would be useful for identifying a vein having a suitable venous access site, the determination based at least in part on the patient information; and an output configured to output the score.

The system can include a near infrared light emitter configured to emit near infrared light onto a target area; and a light sensor configured to receive near infrared light from the target area; wherein the at least one processor is configured to produce a near infrared image for display, the near infrared image based in the near infrared light received by the light sensor.

The output can be a display configured to display the score.

The system can include one or more medical instruments configured to perform one or more measurements to produce at least some of the patient information.

The system can include a communication module configured to receive data from one or more medical instruments to produce at least some of the patient information.

### BRIEF DESCRIPTION OF THE DRAWINGS

FIG. 1 shows a schematic example of an ultrasound system used to generate and display ultrasound images.

FIG. 2A shows an example of an embodiment where a display module is worn as part of a helmet.

FIG. 2B shows an example of an embodiment where a display module is worn as part of a frame as for glasses.

FIG. 2C shows an example of an embodiment where a display module is worn as part of a headband.

FIG. 2D shows an example of an embodiment where a display module is attached to an articulated arm.

FIG. 2E shows an example of an embodiment where a display module is attached to an articulated arm.

FIG. 3 shows an example embodiment of an ultrasound system that includes a heads up display module.

FIG. 3A shows an example of a view of a heads up display module with an ultrasound probe where the displayed image is configured to overlay the imaging target.

FIG. 4A shows a schematic example of an ultrasound system where an ultrasound probe wirelessly communicates data with an image processor.

FIG. 4B shows a schematic example of an ultrasound system where an image processor wirelessly communicates ultrasound images with a display module.

FIG. 5A shows a schematic example of an ultrasound system where an ultrasound probe wirelessly communicates data with both a local image processor and a remote image processor.

FIG. 5B shows a schematic example of an ultrasound system where an image processor wirelessly communicates with both a local display module and a remote display module.

FIG. 6A shows a schematic example of a medical display system where an ultrasound probe and a light sensor both produce images of a single target and both images are displayed on a single display module.

FIG. 6B shows a schematic example of a medical display system where an ultrasound probe, a light sensor, and/or a third sensor can provide input regarding a single target and the information from the three inputs can be displayed on a single multi-input display module.

FIG. 6C shows an example embodiment of a medical imaging system.

FIG. 7 shows a flow chart of an example embodiment of a method for displaying one or more images on a display module.

FIG. 8 shows a flow chart of an example embodiment of a method for recording an attempt to locate a vein with NIR Light.

FIG. 9 is a flowchart of an example embodiment of a method for accessing a patient's vasculature.

FIG. 10 illustrates and example embodiment of a near infrared imaging assessment.

### DETAILED DESCRIPTION OF CERTAIN EMBODIMENTS

Medical imaging technology can enable doctors, emergency medical technicians, nurses, and any other healthcare professionals to examine patients. Ultrasound imaging can be useful to a healthcare professional desiring to know the subcutaneous features in a target area or body portion of a patient. Once a healthcare professional identifies an area to be imaged with an ultrasound system it can be beneficial for the healthcare professional to have quick and convenient access to the ultrasound image. Ultrasound systems can in some embodiments provide a subcutaneous cross sectional view of a target area, which can be a target body portion of a patient. Additionally, in many situations a healthcare professional may need to devote attention to a variety of needs for a single patient, including performing an ultrasound. Some embodiments of this disclosure allow a healthcare professional to quickly and effectively view an ultrasound image while being able to maintain attention and focus to a variety of patient needs.

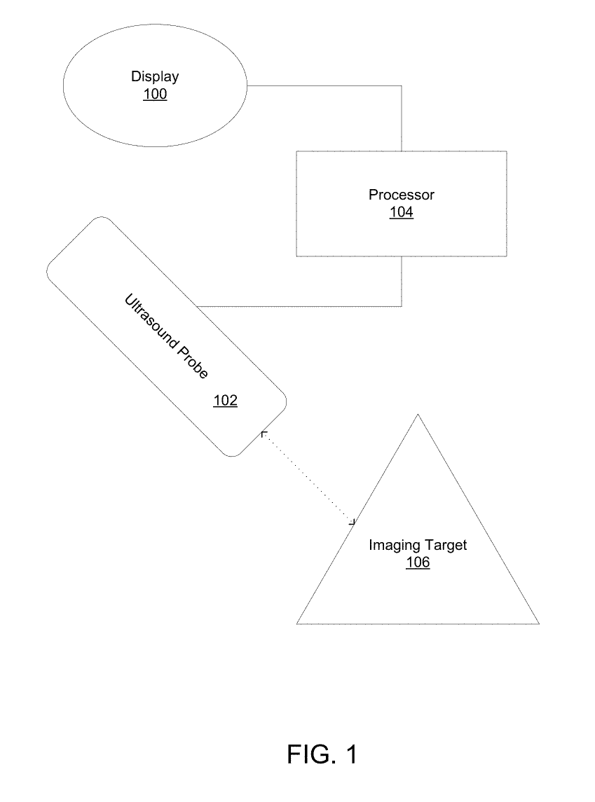

FIG. 1 shows a schematic example of an ultrasound system. In order to display an ultrasound image, an ultrasound probe 102 can send ultrasound signals to an imaging target 106. Imaging target 106 can be a target body portion of a patient or in some embodiments can be any target area. The ultrasound signals' interaction with the imaging target 106 can create reflected echo signals, which can be detected by ultrasound probe 102. Based on the reflected echo signals and initial ultrasound signals the image processor 104 can generate an ultrasound image. This ultrasound image can then be displayed on a display (e.g., display module 100). In some embodiments a doctor or healthcare professional can use the ultrasound image to assist in making a diagnosis or implementing a treatment.

Image processor 104 can develop an ultrasound image in a variety of methods. In some embodiments image processor 104 can use the difference between the ultrasound signal emitted by ultrasound probe 102 and the reflected echo signal to determine the location and density of features (e.g., body tissue) within imaging target 106. Imaging processor 104 can compile reflected echo signal data from a plurality of lines or planes to generate an ultrasound image. In some embodiments, the image processor 104 can be a processor that handles multiple aspects of the ultrasound image system. Image processor 104 can be, in some embodiments, part of the ultrasound probe 102. In some embodiments image processor 104 can also be part of the display module 100. Image processor 104 can be a hardware processor, in some embodiments. The image processor 104 can be implemented as a software program or algorithm, which in some cases can be implemented on a general purpose computer processor.

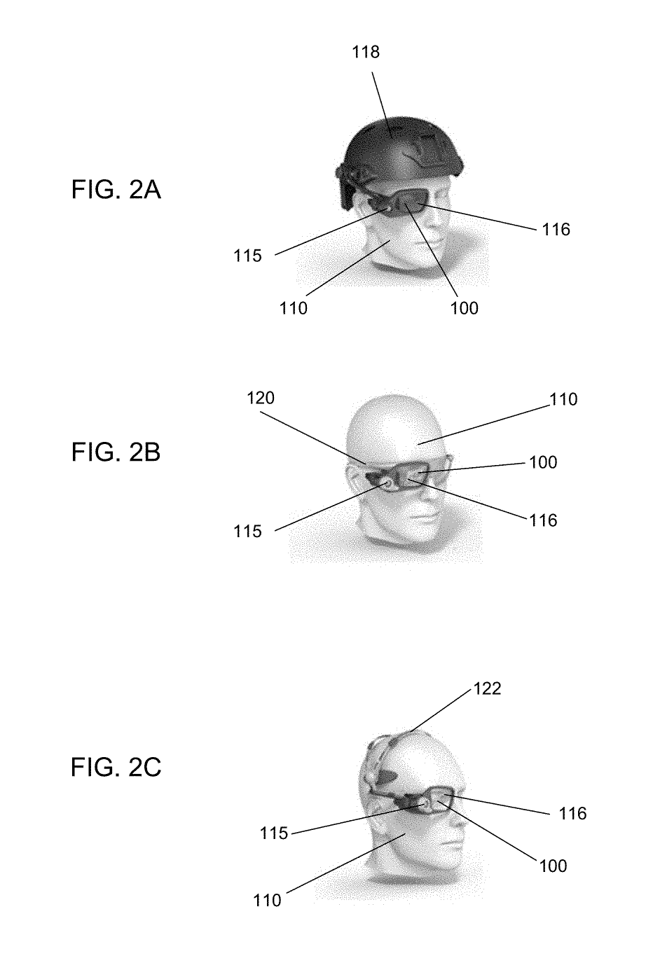

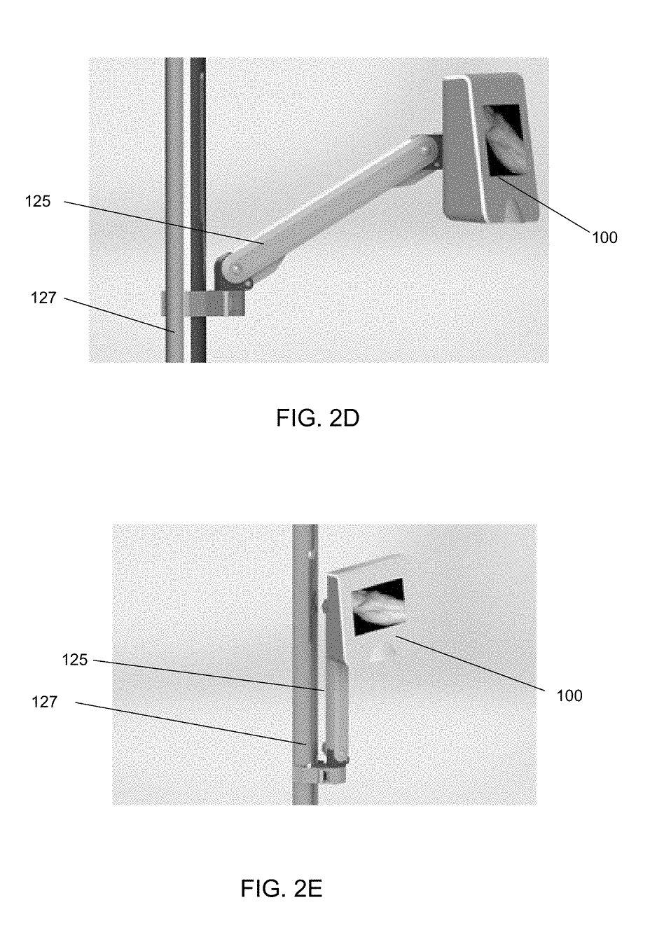

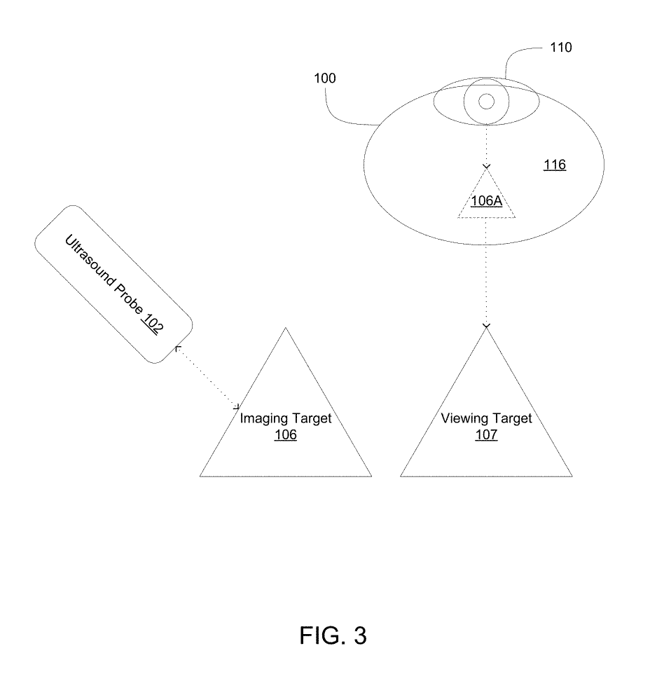

The display module 100 can be wearable, in some embodiments. A wearable display module 100 can produce an image visible to a user (e.g., an operator of the ultrasound system) wearing the display module 100 or wearing structure that supports the display module 100. FIGS. 2A, 2B and 2C show some example embodiments of wearable display modules 100. A wearable display module 100 can be worn on any part of a user 110 such as a head, arm, shoulder or chest. FIG. 2A shows an example of some embodiments where a display module 100 can be worn as or with a helmet 118. FIG. 2B shows an example of some embodiments where a display module 100 can be worn as or with a frame 120 as for glasses or other eyewear. FIG. 2C shows an example of some embodiments where display module 100 can be worn as or with a headband 122. When worn as or with a helmet 118, eyewear frame 120, or headband 122, display module 100 can, in some embodiments, be configured to display an image appearing to be on surface 116. Surface 116 can be configured to be in front of the eye of a user 110 when the display module 100 is worn by a user 110. Surface 116 can in some embodiments be at least partially transparent. Surface 116 can be non-transparent. Many types of displays can be used to present the ultrasound image to the user. For example, in some embodiments, the display 100 can be a direct retinal display.

When wearing a wearable display module 100 in connection with an ultrasound system as shown in FIG. 1, a healthcare professional wearing the display module 100 can locate or adjust the position of the ultrasound image more quickly and easily, compared to having the ultrasound image on a stationary screen that does not move with changes in the user's field of view. For example, in some embodiments a healthcare professional can be the user 110 wearing the display module 100, as shown in FIGS. 2A, 2B, and/or 2C. The display module 100 can be configured to present the ultrasound image in the user's field of view. The display module 100 can be a head-mounted display module 100 or otherwise coupled to the user 110 such that the ultrasound image is maintained in the user's field of view as the wearer's head moves. For example, an image can be displayed on surface 116, which can be maintained immediately in front of the eye of the healthcare professional user 110. Maintaining the ultrasound image in the user's field of view can enable the user 110 to observe the ultrasound image while viewing the target area being imaged by the ultrasound system. The heads up display 100 can also enable the user 110 to observe the ultrasound image while attending to other matters (e.g., looking at another medical device, preparing medication, talking to another medical professional, looking at a body portion of the patient that is not being imaged, etc.). In some embodiments, the ultrasound system can enable the medical professional to attend to multiple needs of a patient in less time, which can be particularly important in an emergency response situation.

As shown in FIGS. 2D and 2E, in some embodiments, the display module 100 can be mountable onto an articulating arm 125, which in some implementations can be slidably coupled to a vertical support member 127. In some embodiments, the height of the articulated arm 125 (and the display module 100) may be vertically adjusted (e.g., by sliding on the vertical support member 127). The display module 100 may be positioned in a wide variety of positions depending on the patient's orientation, the medical practitioner's position, the portion of the patient's body being imaged, etc. In some embodiments, the display module 100 can be mounted onto a point of care cart, onto a clinic utility cart, or onto a variety of other surfaces in various other configurations, for example as disclosed in the '604 Application. In some embodiments, the display module 100 and the support member (e.g., articulated arm 125) can be coupled together by a quick release mechanism that allows a user to quickly release the display module 100 from the support member (e.g., articulated arm 125). Many variations are possible.

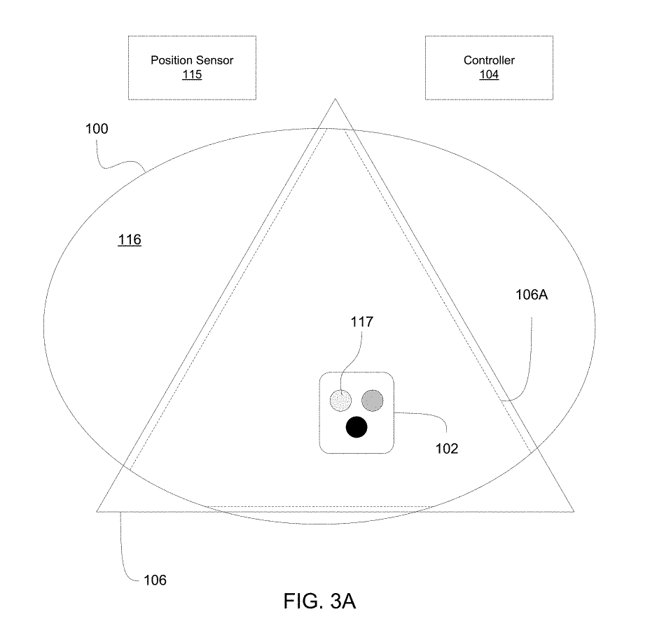

In some embodiments display module 100 can be configured to display images as heads up display images. A heads up display can be any display that presents images that can be viewed without the viewer having to look away from some usual viewpoints. For example, a heads up display can project an image onto an at least partially transparent surface 116. A heads up display can also include a direct retinal display, where an image is projected onto the retina of the eye of a user. FIG. 3 shows an ultrasound system with a heads up display module 100 configured to display an ultrasound image 106A as a heads up display image. In some embodiments, a heads up display module 100 displays an image 106A on a transparent or at least partially transparent surface 116. The surface 116 (e.g., as shown in FIGS. 2A, 2B, and 2C) can be transparent or at least partially transparent.

As shown in FIG. 3, a user 110 can view an ultrasound image 106A of imaging target 106 on the heads up display module 100 while still being able to maintain visual contact with a viewing target 107. The viewing target 107 can be the imaging target 106, the ultrasound probe 102, a different medical device (not shown), another medical professional, etc. When viewing a heads up display module 100 showing an ultrasound image 106A, a healthcare professional user 110 can have the advantage of being able to maintain visual contact with an imaging target 106 on a patient (or other visual targets important to treating the patient) while still viewing the ultrasound image 106A. This can facilitate both more effective use of ultrasound probe 102, and improved ability to respond to multiple healthcare needs of a patient.

In some embodiments, the display module 100 can be attachable to the user 100 at a variety of places, such as the arm, chest, head, or shoulder of the user 110. A wearable heads up display module 100 can be configured to present an ultrasound image 106A for a user 110 to view on a transparent or partially transparent surface 116. A wearable heads up display module 100 can be head-mounted. In some embodiments, the heads up display module 100 can be worn as part of a helmet 118, as part of a frame 120 as for glasses, or as part of a headband 122. The display module 100 as shown in FIG. 2A, 2B, and 2C can in some embodiments be a heads up display module 100. If a heads up display module 100 is worn on the head of a user 110, the user can maintain a view of the image 106A regardless of the orientation of the user's head.

In some embodiments, a head-mounted heads up display module 100 can be configured to display an image appearing to be on an at least partially transparent surface 116 positioned such that when the heads up display module 100 is worn by a user 110 the surface 116 is near the eye of a user 110 and intersecting a straight ahead line of sight of the user 110. In this way, a user 110 would be able to continuously monitor both the image 106A and other important aspects of a patient during examination. This is particularly beneficial during emergency response patient treatment. In some embodiments, the surface 116 can be positioned to be substantially geometrically normal to a straight ahead line of sight for a person 110 wearing the wearable heads up display module 100. In some embodiments, the angle between a line normal to the surface 116 and the straight ahead line of sight can be 30 degrees or less, 15 degrees or less, or 5 degrees or less.

The heads up display module 100 can display images visible at a variety of distances. In some embodiments the heads up display module 100 can be configured to display an image onto a surface 116 that is located near the eye of a user 110. To be located near the eye of a user 110 can mean to be located within the distance that a lens in a frame for glasses would be located relative to the eye of a person wearing the glasses. In some embodiments, the display 100 can produce a virtual image viewable to the user (e.g., by projecting light directly on to the retina of the user's eye or by projecting light on the surface 116 to produce the virtual image). A retinal projector can be positioned near the user's eye. For example, the surface 116 or retinal projector can be positioned at a distance that is at least about 10 mm, at least about 25 mm, at least about 50 mm, or at least about 100 mm from the eye, and/or the surface 116 or retinal projector can be positioned at a distance that is less than or equal to about 300 mm, less than or equal to about 150 mm, less than or equal to about 100 mm, less than or equal to about mm, less than or equal to about 25 mm, or less than or equal to about 15 mm from the eye.

Heads up display module 100 can display the image 106A in a variety of ways. In some embodiments, image 106A can be produced by the projection of light on to an at least partially transparent surface 116 of the heads up display module 100. In some embodiments an image 106A can be scanned directly onto the retina of a user 110.

The ultrasound system can be configured to provide the image 106A as an overlay of imaging target 106. For example, the image 106A can be positioned such that it appears to be positioned over the imaging target 106. FIG. 3A shows an example of some embodiments where displayed image 106A is oriented to overlay the imaging target 106. In some embodiments, image 106A is an ultrasound image, and imaging target 106 is a body portion of a patient. A controller 104 (e.g., the image processor) can be configured to generate the image 106A based at least in part on the position of the ultrasound probe 102 (e.g., based at least in part on the position of the ultrasound probe 102 relative to the display module 100). If the user moves the ultrasound probe 102, a different portion of the target area can be imaged by the ultrasound probe 102, and the controller 104 can be configured to adjust the location of the image 106A on the display 100 such that the image 106A remains positioned over the target area 106 being imaged. If the user moves the display 100 (e.g., by moving user's head while the head-mounted display 100 is attached thereto), the controller can adjust the location of the image 106A on the display 100 such that the image 106A remains positioned over the target area 106 being imaged. The controller 104 can increase or decrease the size of the image 106A to compensate for the display being moved closer to or further from the imaging target 106.

The system can include a position sensor 115, which can be configured to detect the position of the ultrasound probe 102 (e.g., relative to the display 100). The position sensor 115 can include a light sensor (e.g., a visible light sensor or camera). For example, with reference to FIGS. 2A, 2B, and 2C a light sensor 115 can be supported by the head-wearable article (e.g., the helmet 118, the eyewear frame 120, or the headband 122). The light senor 115 can be configured to image an area in front of the user's eyes and/or in front of the display module 100. The light sensor 115 can be coupled to the user 110 such that the light sensor 115 moves with the user 110 (e.g., with the user's head). One or more position indicators 117 can be located on the ultrasound probe 102. The position sensor 115 can detect the one or more positions of the position indicators to determine the position of the probe 102 (e.g., relative to the light sensor 115 and/or display 100). In some embodiments, the position indicators 117 can include dots, colored markings, lights, or other visual indicators. The light sensor 115 can detect the visual position indicators 117 for determining the position of the probe 102 (e.g., relative to the light sensor 115 and/or display 100). In some embodiments, image data from the light sensor 115 can be transmitted (e.g., wirelessly or via a wire) to a controller 104, and the controller can analyze the image data to identify the visual position indicators 117. By comparing the relative positions of the visual position indicators in the image data from the light sensor 115, the position of the ultrasound probe 102 relative to the light sensor 115 and/or display 100 can be determined. For example, if the visual position indicators 117 are closer together that can be an indication that the probe 102 is further away from the light sensor 115, and if the visual position indicators 117 are further apart that can be an indication that the probe 102 is closer to the light sensor 102. In some embodiments, two, three, or more position indicators 117 can be used. In some embodiments, the orientation of the probe 102 can be determined by comparing the relative positions of the visual indicators. The position of the probe 102 can include the location of the probe, the distance of the probe from another object (e.g., the display 100 or the user or the light sensor 115), the orientation of the probe 102, etc.

Many variations and additional features are possible. For example, the light sensor can be coupled to the ultrasound probe and the position indicators can be coupled to the display or to the wearable article. In some embodiments, the ultrasound probe 102 can include an accelerometer or other positioning sensor for determining the orientation or position of the ultrasound probe 102. The system (e.g., the controller 104 or sensor 115) can be configured to determine the position of the ultrasound probe 102 relative to the display, relative to a light sensor 115, relative to a user, etc. In some embodiments, the dedicated position indicators 117 can be omitted and the position of the ultrasound probe 102 itself can be determined directly from the image data (e.g., using image or video analysis techniques).

In some embodiments, the display 100 is not a wearable (e.g., head-mounted) display. For example, the display 100 can be a transparent display that is configured to be positioned between the user and the image target 106. The user can view the image target 106 by looking through the display 100, and the display 100 can display an image overlaid over the image target 106. In some embodiments, the controller 104 can generate the image and/or adjust the image based on the relative positions of the ultrasound probe 102, the display 100, and/or the user. For example, if any one of the probe 102, the display 100, and the user moves, the controller 104 can change the image 106A on the display 100 such that the image 106A remains overlaid over the image target 106. The relative positions of the ultrasound probe 102, the display 100, and/or the user can be determined in a manner similar to the discussion of FIG. 3A. For example, a light sensor can be coupled to the user, and the display 100 and/or probe 102 can include position indicators.

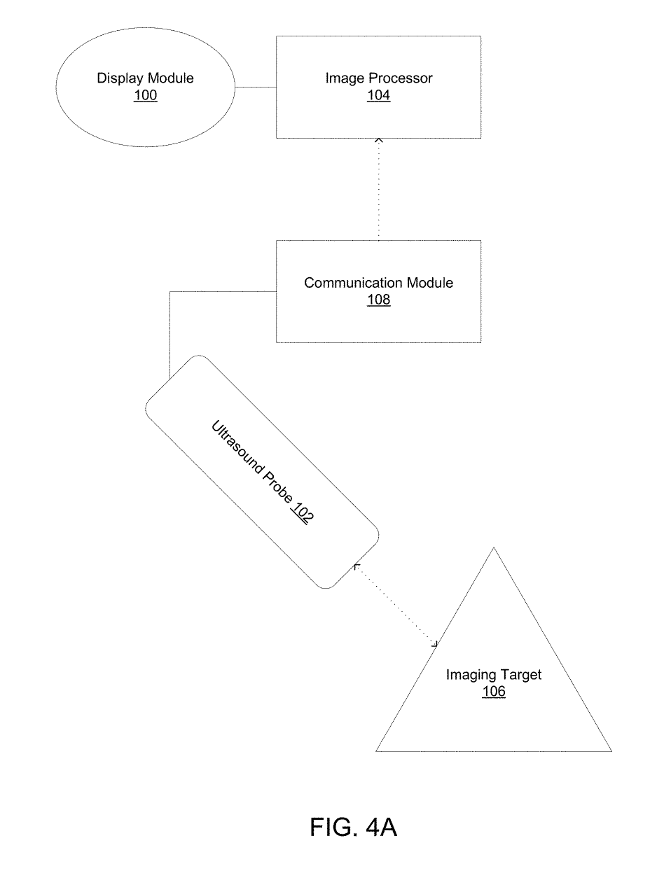

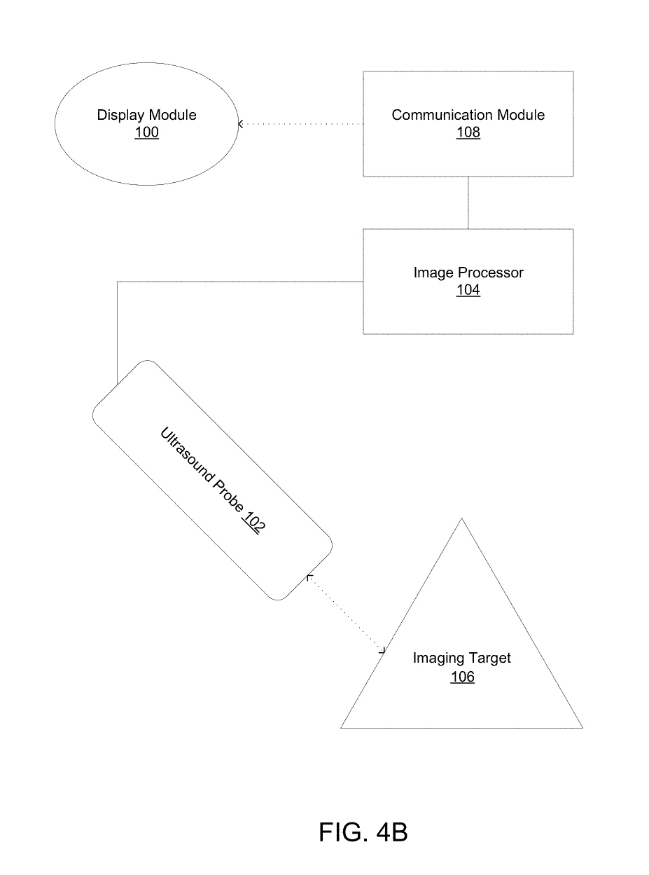

FIG. 4A shows a schematic example of an ultrasound system where an ultrasound probe 102 can send data regarding an ultrasound signal and reflected echo signal from the imaging target 106 wirelessly to an image processor 104. Wireless communication can be achieved through a communication module 108, which in some embodiments may be part of the ultrasound probe 102. The image processor 104 can then generate an ultrasound image of the imaging target 106 to be displayed by display module 100. In some embodiments, the display module 100 and the image processor 104 can be supported by the wearable article. In some embodiments, the wearable article can include a communication module (not shown in FIG. 4A) that can be configured to receive information from the communication module 108. FIG. 4B shows a schematic example of an ultrasound system where an image processor 104 receives data from the ultrasound probe 102 and wirelessly communicates the image to be displayed to display module 100. Wireless communication is achieved through a communication module 108, which can be part of the ultrasound probe 102 and/or part of the image processor 104. The display module 100 may in some embodiments be wearable. Display module 100 may in some embodiments be a heads up display module 100. Although not pictured, in some embodiments, wireless communication can be utilized both between the ultrasound probe 102 and the image processor 104, and between the image processor 104 and the display module 100. In some embodiments, the display module 100 can include or be coupled to a communication module (not shown in FIG. 4B) that can be configured to receive information from the communication module 108. In some embodiments, wires or cables can be used to transfer information between the ultrasound probe 102, the image processor 104, and the display module 100.

The communication modules 108 described herein can provide wireless communication in a variety of ways. In some embodiments, wireless communication can be achieved through a Bluetooth wireless communication link, a Wi-Fi or a wireless local area network (WLAN) communication link, a wireless connection to a cellular system, a commercial communications radio link, a military radio link, or combinations thereof. In some embodiments communication module 108 can include a separate processor from image processor 104. In some embodiments image processor 104 and communications module 108 can involve the same hardware processor. In some embodiments communication module 108 is capable of both sending and receiving information wirelessly. In some embodiments communications module 108 can be a software component. In some embodiments, though not pictured in any figures, additional communication modules may be needed to achieve wireless communication.

Wireless communication at some stage between the ultrasound probe 102 and the display module 100 can allow more freedom to position the display module 100 relative to the imaging target 106, as compared to an ultrasound system that communicates information from the ultrasound probe 102 to the display 100 via wires or cables. A healthcare professional using a display module 100 with wireless communication at some stage between the ultrasound probe 102 and the display module 100 can position the display module 100 (e.g., at a convenient location for effective use of the ultrasound probe 102 and analysis of imaging target 106) without the restrictions or inconvenience of wired connections. In some embodiments, the ultrasound system can utilize both a wearable display module 100 and wireless communication at some stage between the display module 100 and the ultrasound probe 102. In some embodiments an ultrasound system can utilize a heads up display module 100, which may be wearable, as well as wireless communication at some stage between the display module 100 and the ultrasound probe 102. Combining the advantages of a wearable display module 100, a heads up display module 100, and the use of wireless communications can further increase the convenience and flexibility for a user viewing an ultrasound image.

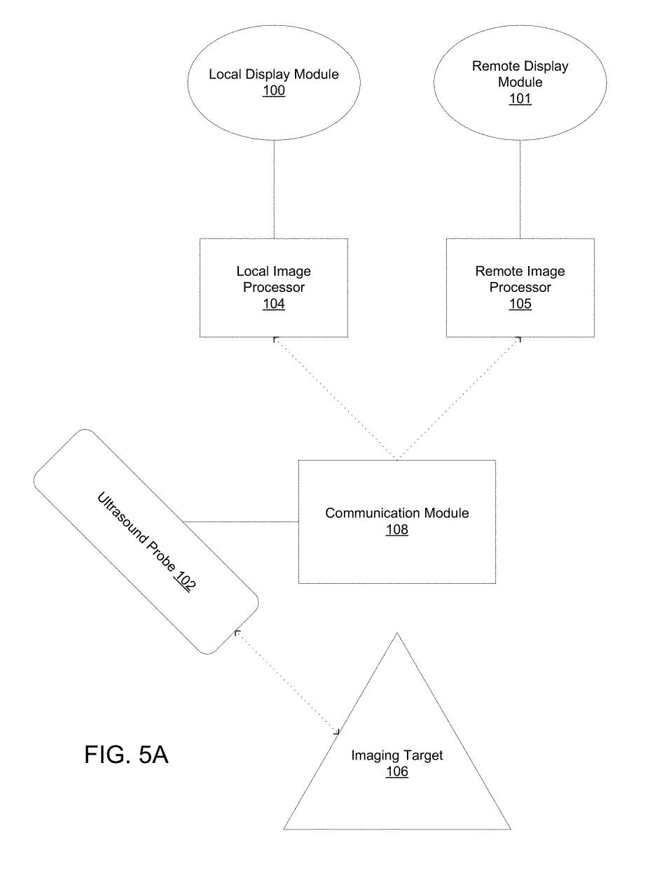

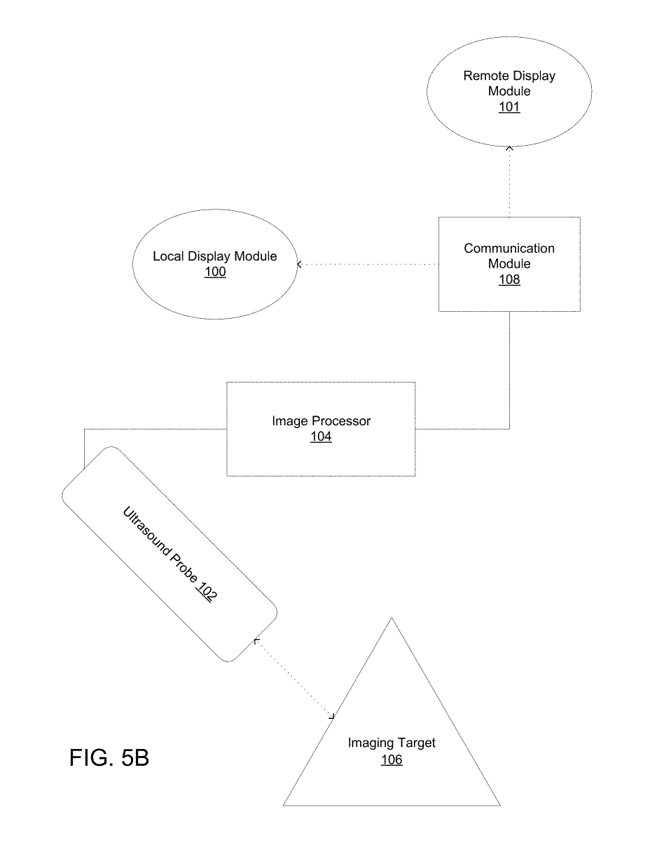

FIG. 5A shows a schematic example of an ultrasound system where information from an ultrasound probe 102 (e.g., the ultrasound signal and reflected echo signal from the imaging target 106) can be transmitted to both a local display 100 and a remote display 101. For example, the information can be transmitted wirelessly to both a local image processor 104 and a remote image processor 105. Wireless communication can be achieved through a communication module 108, which in some embodiments may be part of the ultrasound probe 102. Local image processor 104 can generate an image to be displayed by local display module 100, and remote image processor 105 can generate an image to be displayed on a remote display module 101, which can be accessible to a remote medical professional (e.g., a doctor). Accordingly, the system can enable a local medical professional (e.g., an emergency medical technician) on site with the patient (e.g., at a scene of an accident or emergency) to view the ultrasound image on the local display 100 (e.g., for performing the ultrasound), and can enable a remote medical professional (e.g., a doctor) at a remote location (e.g., a hospital or doctor's office) to view the ultrasound image on the remote display 101 (e.g., for analyzing the ultrasound image, such as for diagnosis).

Local image processor 104 and remote image processor 105 can in some embodiments provide identical images for display in the respective display modules. In some embodiments remote image processor 105 can be configure to deliver a lower quality or higher quality image compared to local image processor 104. If the local display module 100 has different specifications for image display than remote display module 101, then the local image processor 104 and remote image processor 105 can, in some embodiments, provide different images of imaging target 106 to each respective display module in order to meet the different specifications of the display modules. Local image processor 104 may in some embodiments be less sophisticated or powerful than the remote image processor 105. Different images can also be displayed in response to either different processing power or different viewing needs associated with local display module 100 compared with remote display module 101. Although not pictured in FIG. 5A, wireless communication can in some embodiments be used between local image processor 104 and local display module 100, between remote image processor 105 and remote display module 101, or between both.

FIG. 5B shows a schematic example of an ultrasound system where image processor 104 generates an ultrasound image of imaging target 106, and the ultrasound image can be wirelessly communicated by communication module 108 to local display module 100 and to remote display module 101. Although not pictured in FIG. 5B, in some embodiments wireless communication can be utilized between the ultrasound probe 102 and image processor 104. In some embodiments image processor 104 can deliver a single image to both local display module 100 and remote display module 101. In some embodiments image processor 104 can deliver different images of imaging target 106 to the local display module 100 and the remote display module 101. Different images may be delivered in order to satisfy different image display specifications or to meet different viewing needs at the remote display module 101 compared with the local display module 100. For example, if a remote healthcare profession is attempting to provide treatment or diagnostic instructions to a local user imaging the imaging target 106, then the remote healthcare professional may need to view an image in greater detail than the local user. Accordingly, in some embodiments, the image processor 104 can generate a first image for the local display 100 and a second image for the remote display, and the second image can have higher quality (e.g., higher resolution) than the first image.

In the embodiments described in connection with FIGS. 5A and 5B, local display module 100, remote display module 101, or both can be a wearable display module. In some embodiments local display module 100, remote display module 101, or both can be configured to display an ultrasound image as a heads up display, similar to the above discussed heads up display module 100. A local display module 100 may be configured to be a wearable heads up display module 100 in order to facilitate quick and convenient viewing of an ultrasound image, while remote display module 101 may be a non-wearable display (e.g., a desktop display, mobile device display, laptop computer display, etc.). The remote display 101 can be larger than the local display 100, e.g., in order to facilitate more detailed and thorough examination of the ultrasound image in a context where convenience of display is less important.

A remote healthcare professional can access the remote display module 101 and can provide diagnostic or treatment information to a local healthcare professional (e.g., the user wearing the local display 100). By displaying the image in two locations better collaboration and medical decision making can be achieved. In the context of emergency response treatment, a remote doctor viewing an ultrasound image of imaging target 106 can provide medical or diagnostic information to a healthcare professional at the scene of the emergency that may not otherwise be available, an a patient can receive treatment at the scene of the emergency without being transported to a hospital or doctor's office.

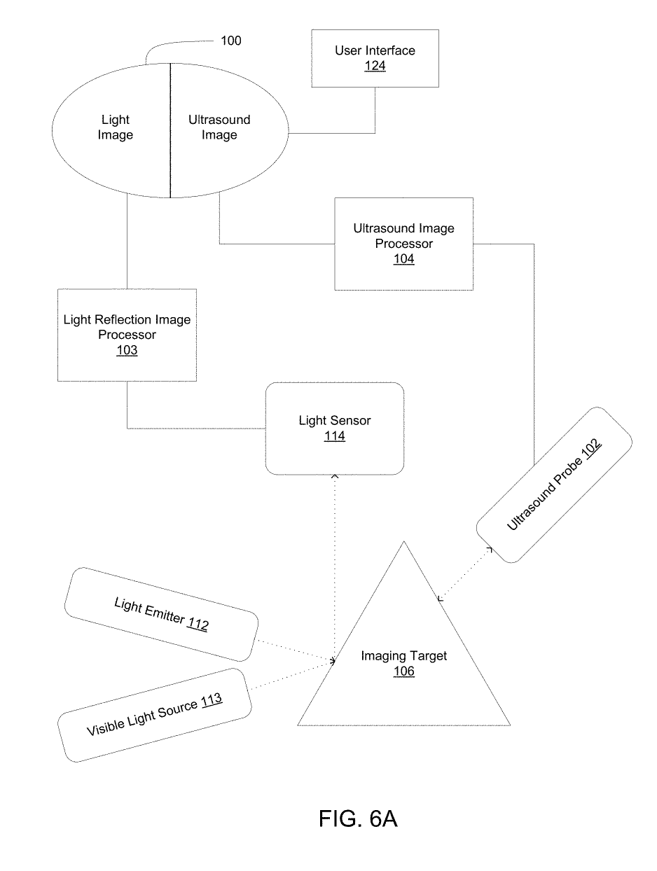

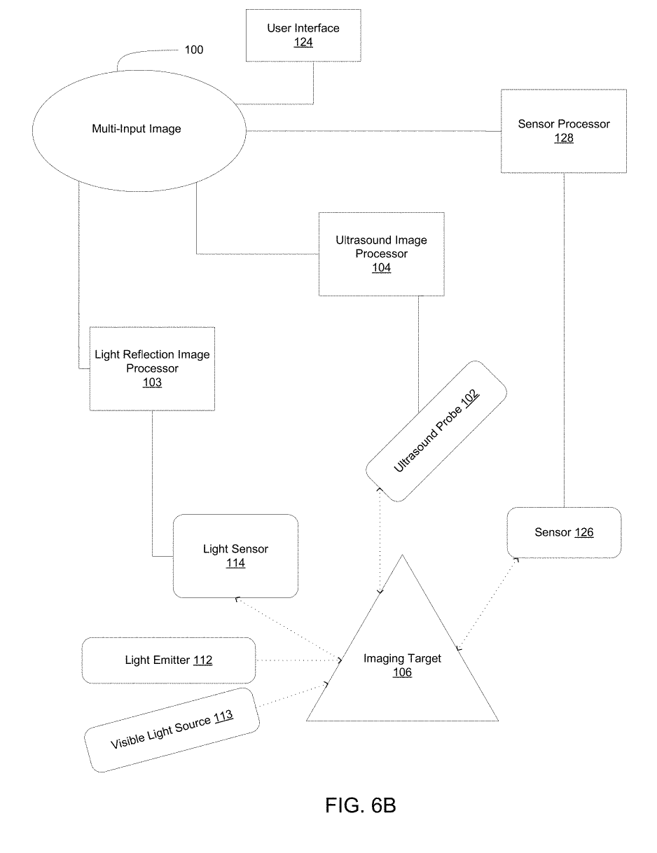

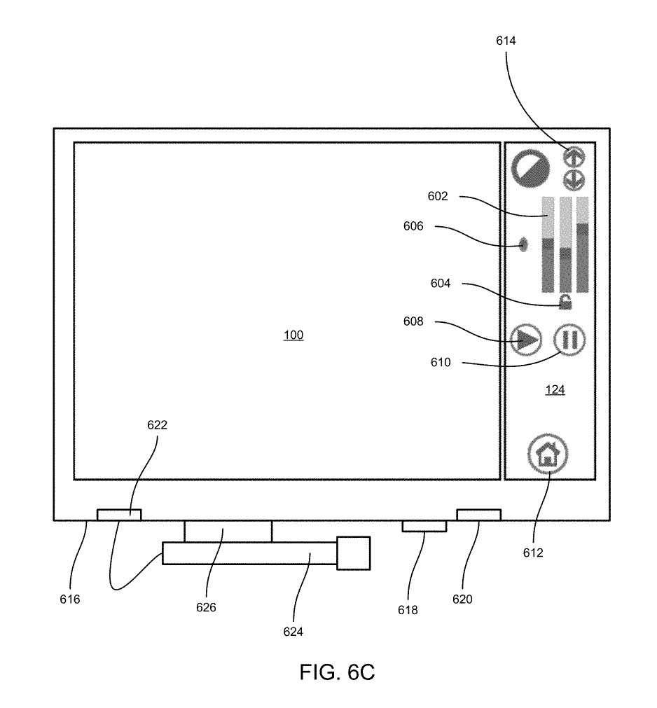

In some embodiments a display module 100 may be configured to display images from multiple imaging sources. The FIG. 6A shows a schematic example of a system where a multi-image display module 100 can display images of imaging target 106 from a light sensor 114 and an ultrasound probe 102. Imaging target 106 can be a target body portion of a patient or any target area.

Ultrasound probe 102 can emit ultrasound signals and can receive reflected echo signals from imaging target 106. Based at least in part on data from ultrasound probe 102, an ultrasound image processor 104 can generate an ultrasound image, which can be displayed on a display 100. Light emitter 112 can emit light, which can be of a specific wavelength, onto the imaging target 112. A light sensor 114 can be configured to detect light emitted by light emitter 112 that is reflected and/or scattered from imaging target 106. Based on data from the light sensor 114, a light image processor 103 can generate a light image indicating the features of imaging target 106 based on the light reflected and/or scattered by the imaging target 106. In some embodiments a light image can be a near infrared light image (NIR image). Display module 100 can then display both the light image and the ultrasound image. In some embodiments, the display 100 can display the ultrasound image and the light image simultaneously (e.g., side-by-side or one above the other). In some embodiments, the display 100 can display the ultrasound image and the light image overlaid one image over the other, or a single image produced from both the ultrasound image and the light image. In some embodiments, a user interface 124 enables a user to select whether the display 100 shows the ultrasound image or the light image.

A light emitter 112 can in some embodiments be configured to emit near infrared (NIR) light. Reflection or scattering of NIR light can be used to show the locations of veins in the body portion of the patient. The light emitter 112 can be configured to emit light between about 600 nm and about 1000 nm, in some embodiments. The light emitter 112 can emit light that is configured to be absorbed by hemoglobin. The light sensor 114 can detect light that was reflected or scattered from imaging target 106 (and thus not absorbed by hemoglobin), and in some embodiments the NIR light image produced by light image processor 103 can illustrate distinct locations of hemoglobin in blood and the surrounding tissue. The NIR light image can be used to identify veins, to check patency of a vein, and/or or to identify infiltration or extravasation, for example as discussed in the '604 Application.

In some embodiments, the light emitter 112 can emit multiple wavelengths of light (e.g., as discussed in the '604 Application). For example, the light emitter 112 can comprise multiple different light emitters of varying types (e.g., LEDs) that are configured to emit different wavelengths (e.g., different lines) of light (e.g., see FIG. 3 of the '604 Application and the accompanying text). Although some embodiments are discussed as having three different light emitter types with three different wavelengths (e.g., three different lines) that produce three different image contributions, any number of light emitter types, wavelengths, and image contributions can be used (e.g., 2, 4, 5, 6, etc.). For example, 2, 3, or 4 types of LED sets can be used to emit light of different wavelengths ranging from about 700 nm to about 1000 nm, and in some embodiments, the LEDs can be pulsed or sequenced, as discussed herein. Various spectral outputs can be used. For example, the light emitters can have nominal wavelengths (e.g., lines or peaks) of about 740 nm, about 850 nm, and about 950 nm respectively. In some embodiments, a first light emitter can emit light having a peak at about 700 nm to about 800 nm (e.g., about 750 nm to about 760 nm). A second light emitter can emit light having a peak at about 800 nm to about 900 nm (e.g., about 850 nm to about 870 nm). A third light emitter can emit light having a peak at about 900 nm to about 1100 nm (e.g., about 940 nm to about 950 nm). In some embodiments, the spectral output of the light emitters can have bell curve (e.g., Gaussian) shapes. In some embodiments, the spectral output curves for the different light emitters can overlap each other. Light from the first light emitter can be used to produce a first image contribution of high quality but that reaches only a short distance into the tissue depth. Light from the second light emitter can be used to produce a second image contribution that has lower quality than the first image but reaches deeper into the tissue than the first image contribution. Light from the third light emitter can be used to produce a third image contribution that is able to reach deeper into the tissue than the first and second image contributions but has a lower quality than the first and second image contributions. In some embodiments some or all of the multiple light emitters can emit light with lines at wavelengths between about 1000 nm and about 2500 nm. In some embodiments, a single broadband NIR light source can be used instead of multiple distinct light source types. Various embodiments discussed herein relate to multiple light sources that emit light having different NIR spectral outputs (e.g., different wavelengths, different lines, and/or different peaks). A spectral line as described herein can relate to a portion of a spectral output that includes a peak and a trough, wherein the intensity of the peak is 2 times greater than the trough, 3 times greater than the trough, 4 times greater than the trough, or more.

In some embodiments, all light emitters comprising the light emitter 112 can be turned on at the same time so that the light from all three light emitters illuminates the target area simultaneously. Light of multiple wavelengths (e.g., multiple lines) can be reflected or scattered by the target area to the light sensor 114 to produce a single composite image that is a combination of the different image contributions. A user can in some embodiments adjust the relative intensity of the different light emitters. If light emitters of different wavelengths are being used in the light emitter 112, a user may opt to increase the relative intensity of a certain light emitter in order to adjust the quality of the image versus the depth of penetration for the image. For example, a user may opt to increase the relative intensity of the light emitter having the largest wavelength in order to produce an image reaching deeper into tissue. Conversely, for example, a user may opt to increase the relative intensity of an emitter having a lower wavelength in order to produce an image with greater quality but lower penetration depth. A user can in some embodiments change the relative intensities through a user interface such as the user interface 124. Increasing or decreasing the relative intensity of a light emitter can include turning one light emitter completely off.

FIG. 6C shows an example embodiment of a medical imaging system. The medical imaging system can include a display module 100 and user interface 124. In some embodiments, the system can include a touchscreen that is configured to display images and operate as the display module 100 and also is configured to receive touch input from a user to operate as the user interface 124. The user interface can include various user input elements. The user interface 124 can include one or more user input elements 602 for adjusting settings of the NIR light source(s) that make up the light emitter 112. For example, if the light emitter 112 includes three NIR light sources, the user interface 124 can include three user input elements 602 (e.g., sliders) for adjusting the intensity of light output by the three light sources. The user interface 124 can include a locking user input element 604 which can receive input to lock or unlock the user input elements 602 (e.g., the sliders) that control the intensity of the light sources. When the locking user input element is unlocked, the relative intensity of the light sources can be adjusted (e.g., by moving the slider user input elements 602). When the locking user input element is locked, in some embodiments, the intensity of the NIR light sources of the light emitter 112 can be adjusted together as a group (e.g., such that adjusting one of the slider user input elements 602 causes all the slider user input elements 602 to move together). The one or more user input elements 602 can be used to adjust various parameters (e.g., duty cycle, frequency modulation, amount of time that each light source is on, and/or brightness) of the NIR light sources to vary the optical illumination provide by the different light sources. In some embodiments, the user interface can include a visible light adjustment input element 606, which can enable the user to adjust settings of the visible light source 113 (e.g., the color and/or intensity). The user interface 124 can include an activation user input element 608, which can cause NIR imaging to start. The user interface 124 can include a pause user input element 610, which can stop the NIR imaging and/or hold the last image captured on the display 100. Various other user input elements can be included to enable the user to perform the various operations disclosed herein and in the '604 Application. By way of one example, the user interface 124 can include a store image input element (not shown in FIG. 6C), which can store an NIR image, as discussed herein. The user interface 124 can include a home or return user input element 612, which can cause the user interface to change to a different interface (e.g., an interface with user input elements for selecting NIR and/or ultrasound imaging). The user interface 124 can include one or more display adjustment input elements 614 for adjusting settings of the display 100 (e.g., contrast, brightness, color, etc.).

The medical imaging system can include a housing 616, which can support the display 100 and/or other components of the system. The system can include one or more light sources 618 (e.g., the NIR light emitter(s) 112 and/or the visible light source 113) and a light sensor 620 for NIR imaging. The system can include one or more input modules 622 (e.g., a wireless input module or an electrical port such as a USB port) configured to receive input from a medical instrument (e.g., an ultrasound probe 624, spectrometer, etc.) as discussed herein. The system can include a coupling mechanism 126 configured to mechanically couple the medical instrument (e.g., the ultrasound probe 624) to the housing 616. The coupling mechanism 126 can be a clip, a pocket, a slide engagement, etc. The coupling mechanism 126 facilitate rapid and easy transition from using the NIR imaging system (e.g., the light source 618 and sensor 620) to using the medical instrument (e.g., the ultrasound probe 624).

In some embodiments, the light emitters can be pulsed in sequence with the light sensor 114 (e.g., synchronized with a shutter of the camera), so that the light emitters are turned off when the light sensor 114 is not generating an image and so that the light emitters are turned on when the light sensor 114 is generating an image. In some cases, the pulsing of the light emitters can be synchronized with the shutter of the camera so that the light emitters are turned on when the shutter is open and turned off when the shutter is closed. Turning the light emitters off when not needed can reduce power usage and heat buildup. In some embodiments, a light emitter 112 that includes only a single light emitter, or light emitters of all substantially the same wavelength, or of different wavelengths, can be pulsed at a rate that corresponds to an imaging rate of the light sensor 114.

In some embodiments, the light emitters can be pulsed sequentially. For example, at a first time, the first light emitter can be turned on while the second and third light emitters are turned off, and the light sensor 114 can generate a first image at the first time using the light from the first light emitter. At a second time, the second light emitter can be turned on while the first and third light emitters are turned off, and the light sensor 114 can generate a second image at the second time using the light from the second light emitter. At a third time, the third light emitter can be turned on while the first and second light emitters are turned off, and the light sensor 114 can generate a third image at the third time using the light from the third light emitter. Additional images can be generated by additional light emitters of different wavelengths, depending on the number of different wavelengths utilized. In some embodiments, the system can perform different image processing on the different images that correspond to the respective different wavelength lines (e.g., from the different light sources). For example, in some embodiments, a first image processing procedure (e.g., a filter or look-up-table conversion) can be performed on the first image that is optimized or otherwise configured for use with the spectrum of light output by the first light source. A second image processing procedure (e.g., a filter or look-up-table conversion) can be performed on the second image that is optimized or otherwise configured for use with the spectrum of light output by the second light source. A third image processing procedure (e.g., a filter or look-up-table conversion) can be performed on the third image that is optimized or otherwise configured for use with the spectrum of light output by the third light source. By way of example, the look-up-table conversion can convert the raw pixel values from the imaging sensor into a different set of values (e.g., for use in the displayed image). The look-up-table can remap the grayscale levels of the pixels or colorize the image, for example. The different images can be displayed on the display module 100 in rapid succession (e.g., interlaced) so that the images combine to form a composite image of all three images to the human eye. Similarly, the different images can be stored in memory and then combined by the imaging system to form a composite image, which may be displayed on the display module 100 to the user. Optionally, a control may be provided enabling the user to instruct the imaging system via a user interface 124 to display each image individually and/or to display a composite image including images selected by the user.

Pulsing the light emitters sequentially can allow for more light of each wavelength to be used. For example, if all three light emitters are turned on together, the amount of light emitted by each light emitter may need to be limited or reduced to avoid overpowering the light sensor 114. However, if the light emitters are pulsed sequentially, more light of each wavelength can be used since the light is not combined with the other wavelengths of light from the other light emitters. By illuminating the target area with more light of each of the three light emitters, the quality and/or imaging depth of the produced image can be improved. In some sequentially pulsing embodiments, the light sensor 114 can be configured to capture images at a faster rate (e.g., 60 hz or 90 hz) than would be needed in embodiments in which the light emitters are turned on together, since the different image portions are captured separately. In some embodiments, the light sensor 112 can include multiple light sensor portions (e.g., as subpixels of the light sensor 112) configured to synchronize with the multiple light emitters that are pulsed in sequence. In some embodiments, different light sensors can be used for the different wavelengths of light and can be configured to synchronize with the pulsing of the multiple light emitters.

The composite image that includes the three image portions can provide the benefits of all three image portions to the user simultaneously, without requiring that the user toggle between the different wavelengths of light. When the user wants to observe a feature that is relatively deep in the tissue, the user can focus on the third image portion of the composite image, which is produced using the longer wavelength NIR light. When the user wants to observe high quality detail of a feature that is relatively shallow in the tissue, the user can focus on the first image portion of the composite image, which is produced using the shorter wavelength NIR light. Although the presence of the third image portion can degrade the quality of the first image portion to some degree, it is expected that the human mind is able to focus on the desired portions of the image while deemphasizing the other portions of the image. Various embodiments disclosed herein can utilize a light emitter 112 that is configured to pulse, as discussed herein, and can include multiple light emitters for producing images with different wavelengths of light, even where not specifically mentioned in connection with the specific embodiments.

In some embodiments a visible light source 113 can be incorporated in the imaging system. The visible light source 113 can produce visible light that illuminates the same general area as the light emitter 112 (e.g., the imaging target 106). In some embodiments the visible light source 113 provides visible illumination of the imaging target 106 to better facilitate inspection or interaction with the imaging target 106, such as insertion of a needle at the imaging target 106. The visible light source 113 can also provide a visible cue that other potentially non-visible light is being emitted by light emitter 112. Absent any visible light, non-visible light (e.g., near infrared light) from the light emitter 112 could potentially shine continuously in a person's eye without that person noticing the exposure. Thus, a visible light source 113 could provide a desirable safety feature. The visible light from the visible light source 113 can be of any color or wavelength, and in some embodiments the light is green, blue, red, white, etc. In some embodiments the visible light source 113 can emit broadband (e.g., white) visible light. In some embodiments the visible light source 113 can be a colored light source (e.g., a red or orange light source), which can emit visible light having a wavelength (e.g., red or orange light) that is configured to facilitate the identification of blood vessels (e.g., veins) in the imaging target 106 (e.g., in the body tissue of a patient) with the naked eye. For example, the visible light can have a wavelength (e.g., red or orange light) that is absorbed more by the blood vessels (e.g., by the hemoglobin in the blood) than by the surrounding tissue, such that illumination of the target area with the visible light can facilitate the identification of the blood vessels (e.g., veins), for example, with the naked eye.

Although various embodiments are disclosed in connection with accessing a patient's vein (e.g., by inserting a needle or other medical implement into the vein) and/or imaging a patient's veins, the systems and method can also be applied to imaging and accessing other blood vessels as well.

The capability in some embodiments to present multiple medical images for viewing on a single display module 100 can provide a healthcare professional with improved ability to analyze an imaging target 106. A light image utilizing near infrared (NIR) light can provide a healthcare professional with a view of veins in imaging target 106. An ultrasound image can show a subcutaneous cross-sectional view of imaging target 106. When viewed together or in sequence, the NIR light image and the subcutaneous cross-sectional view (e.g., ultrasound image) of the imaging target 106 can provide complimentary information to a healthcare professional. By providing healthcare professionals with improved access to multiple medical images from different sources, a medical imaging system can facilitate quicker action by healthcare professionals. Accordingly, some embodiments can be particularly valuable in an emergency response context where time is of the essence.

In some embodiments, the ultrasound image can be used to calibrate and/or validate the NIR light image. The combined use of an ultrasound image in addition to a light image (e.g., an NIR light image) can in some embodiments be used to determine the inner diameter and outer diameter of a blood vessel. The light image can show the transverse path of a blood vessel (e.g., along a generally planar region that is generally perpendicular to the direction of the light emitted from the light emitter 112 onto the imaging target 106). One or more ultrasound images can provide a cross sectional image of the blood vessel (e.g., along a direction that can be generally perpendicular to the generally planar region shown in the light image). The inner diameter and/or outer diameter of the blood vessel can then be determined from one or more ultrasound images. For example, in some embodiments, the system can enable the user to identify two or more locations associated with the vein (e.g., the outer boundaries defining the outer diameter of the vein or the inner boundaries defining the inner diameter of the vein) on the ultrasound image, and the system can be configured to determine the physical diameter based at least in part by the two or more locations identified by the user. In some embodiments, the system can be configured to automatically identify the inner and/or outer boundaries of the vein in the ultrasound image (e.g., using edge detection), and the system can automatically determine the inner diameter and/or outer diameter of the vein. Knowing a blood vessel's inner and outer diameters can be useful to a medical professional to assess the vein and to select the proper catheter. In some embodiments, the system can be configured to provide a recommendation of an appropriate catheter or appropriate catheter size based at least in part on the diameter of the vein.

In some embodiments light imaging (e.g., the NIR imaging) can be used to determine the blood vessel's inner diameter and/or outer diameter without using an ultrasound image. For example, the system can enable the user to identify the inner and/or outer boundary of the vein. In some embodiments, edge detection algorithms can be used on the light image to identify the inner and/or outer boundaries of the vein in the NIR image. The system can be configured to determine the inner diameter and/or outer diameter of the vein from the identified inner and/or outer boundaries identified in the NIR image. In some embodiments, a reference object (e.g., a needle or other object) can be used to facilitate the size determinations (e.g., vein size) made by the system. The reference object can have a predetermined size, or it can have markers that are space apart by a predetermined distance, and the reference object can be positioned such that it will be imaged by the system so that the image of the reference object can be used by the system to make size determinations of other objects that are being imaged. In some embodiments, multiple imaging methods can be used together to determine size measurements (e.g., inner diameter and/or outer diameter of a vein), such as NIR imaging and/or ultrasound imaging.

The display module 100 can display more than one image at a single time. In some embodiments, the multi-image display module 100 may display both a light image (e.g., an NIR image) and an ultrasound image of a single imaging target 106 in close proximity at a single time. A user viewing an image on the multi-image display module 100 can, in some embodiments, alter the portion (e.g., the ratio) of the display 100 attributed to any displayed image (e.g., by proving input via the user interface 124). For example, the system can be configured to respond to user input received by the user interface 124 to change the output of the display 100 from a relatively large NIR image to a relatively small NIR image and/or from a relatively small ultrasound image to a relatively large ultrasound image. In some embodiments multiple images can be viewed in sequence on the multi-image display module 100, and in some embodiments a user can control what image is displayed at a given time (e.g., via input provided to the user interface 124). In some embodiments, the multi-image display module 100 can display a single image at a time. The display module 100 can display a light image and an ultrasound image side-by-side or one above the other. The display module 100 can display the light image and the ultrasound image overlaid one over the other, or a single composite image generated from both the light image and the ultrasound image. Multiple images of an imaging target 106 from multiple inputs such as from a light sensor 114 and an ultrasound 102 may be overlaid to display a composite image of target 106. A user can in some embodiments select through user interface 124 whether to display input from multiple sources as a single overlaid image or as separate images in close proximity (i.e. side-by-side). In some embodiments a user can select the focus or balance between overlaid images.

The display module 100 can, in some embodiments, display other types of medical images (e.g., at a single time) such as X-Ray images or MRI images, or other types of medical data such as a patient's temperature, heart rate, oxygen saturation, blood pressure, etc. Additional images or medical data can in some embodiments be displayed in combination with an ultrasound image, a light image, both, or neither. In some embodiments, a multi-image display module 100 can display three or four or more medical images in close proximity at a single time, or at different times (e.g. in response to user input). As shown in FIG. 6B, in some embodiments a sensor 126 can be incorporated in a medical imaging system. The sensor 126 can be connected to a sensor processor 128, which transforms the information collected by sensor 126 into an image or readable data to be displayed by the display module 100. While only a single additional sensor 126 is pictured in FIG. 6B, in some embodiments two or more sensors 126 can be incorporated in the imaging system in combination with a light reflection sensor, an ultrasound probe, both, or neither.

The sensor 126 can be any sensor that provides medical data or imaging. In some embodiments sensor 126 can be the leads for an electrocardiogram (EKG or ECG). In such embodiments, the output from the EKG leads can be processed by the sensor processor 128 and provided on the display module 100 (e.g., in combination with a light reflection image, an ultrasound image, both, or neither). In some embodiments the sensor 126 can be a spectrometer, which can detect information from a target area based on the spectrum of light reflected from the target area. For example, the spectrometer can provide information regarding the chemical composition of the target area 106 based on the spectrum of light that is returned to the spectrometer. In some embodiments, the spectrometer can include one or more light sources configured to emit light (e.g., broadband white light) onto the imaging target 106 such that the emitted light can be reflected from the imaging target to be received by the sensor 126 of the spectrometer. The system can automatically turn off the light emitter 112 and/or the visible light source 113 during operation of the spectrometer that includes its own light source. In some embodiments, the spectrometer can be configured to operate by receiving light from the light emitter 112 (e.g., NIR light) and/or light from the visible light source 113 that is reflected from the imaging source 106 onto the sensor 126. In some embodiments, the system can be configured to use different settings (e.g., intensities) for the light emitter 112 and/or visible light source 113 during NIR imaging than during operation of the spectrometer, and the system can be configured to automatically change the settings (e.g., intensities) of the light emitter 112 and/or visible light source 113 depending on whether the system is operating the NIR imaging system or the spectrometer. In some embodiments, the data provided by the spectrometer can depend on the output of the light emitter 112 and/or visible light source 113, which can be adjustable by the user. In some embodiments, the system can be configured to override user commands relating to the light emitter 112 and/or the visible light source 113 when the sensor 126 of the spectrometer is collecting light. For example, if the user sets custom intensities for NIR imaging using the three light sources that make up the light emitter 112, the system can be configured to use the custom intensities for the three light sources during NIR imaging, and the system can change one or more of the intensities of the three light sources to values adapted for use with the spectrometer during operation of the spectrometer. In some instances, the NIR imaging and the spectrometer operation can be performed in rapid succession with the light intensities changing rapidly between the NIR imaging settings and the spectrometer settings, such that the user perceives that the NIR imaging and spectrometer operation occur simultaneously. In some embodiments, the spectrometer can operate while the light from the light emitter 112 and/or the visible light source 113 are on, and the processing of the data from the spectrometer can compensate for the light from the light emitter 112 and/or the visible light source 113. In various embodiments, the spectrometer can comprise a wavelength dispersion element such as a diffraction grating or a prism that disperses light into different wavelengths that can be detected by a light sensor or light sensor array to determine the spectral composition of the received light. Further example embodiments can include the one or more sensors 126 comprising part of: an X-Ray emitter and detector; a magnetic resonance imaging (MRI) device; a pulse oximeter; a blood pressure monitor; a digital stethoscope; a thermometer; an otoscope; and/or an examination camera.

In some embodiments wireless communication can occur between the sensor 126 and the sensor processor 128, or among the sensor 126, the sensor processor 128 and any other component of the imaging system. The system can include a wireless input module configured to receive input wirelessly from the sensor 126. In some embodiments, the ultrasound probe 102 and/or the sensor 126 (e.g., the EKG or ECG device, the spectrometer, the X-Ray device, the MRI device, the pulse oximeter, the blood pressure monitor, the digital stethoscope, the thermometer, and/or the otoscope) can be coupled to the system by a detachable cable (e.g., via an input module such as a USB port), for example, as disclosed in the '604 Application. Accordingly, the system can provide a single, portable, modular system that can be a platform for various types of medical imaging and medical data collection and display.

In some embodiments the system can be used to facilitate insertion of a peripherally inserted central catheter (PICC). A PICC can be inserted in a patient's peripheral vein and then advanced toward the patient's heart until the tip rests in the distal superior vana cava, cavoatrial junction, or other suitable location. The location of the tip can be confirmed by X-Ray imaging or by a signal from an EKG. An ultrasound image, a light reflection image (e.g., NIR image), or both can be used to facilitate location and insertion of a PICC into a peripheral vein. In some embodiments a sensor 126 can be configured to confirm that the tip of the PICC has reached the desired location, for example the sensor 126 can be a set of EKG leads or an X-Ray emitter and detector such as used in fluoroscopy. The system can provide a single imaging system to facilitate both the initial insertion of the catheter into the peripheral vein and the advancement of the catheter to the desired location in the patient's body.

The display module 100 can display medical images or data from multiple sources in a variety of manners, such as: in sequence, in close proximity on a single screen or on multiple screens, substantially overlaid onto a single image, or any combination thereof. In some embodiments a user can select through the user interface 124 which medical images or data to display on display module 100, and in which manner. For example, a user could select to display an overlaid ultrasound image and light reflection image (e.g., NIR image) with the patient's temperature and readings from an EKG both displayed in close proximity to the overlaid image.

In some embodiments the display module 100 can be coupled to or integrated within a single housing with some or all other components of the system. The housing can in some embodiments encompass one or more of: a display module 100; a user interface 124; a light reflection image processor 103; a light sensor 114; a light emitter 112; a visible light source 113; an ultrasound image processor 104; a port for coupling an ultrasound probe 102; one or more sensor processor 128 such as a processor for an EKG, a spectrometer, or other sensor; and/or one or more ports for receiving one or more medical devices, such as one or more sensors 126 such as an EKG lead input, a spectrometer, or other sensors. In some embodiments the display module 100 can be a touchscreen capable of both facilitating the user interface 124 and providing images of the imaging target 106. In some embodiments one or more hardware processors serving as any of the ultrasound processor 104, the light reflection image processor 103, or the additional sensor processor 128 can be integrated with the display module 100 and the user interface 124 in a single housing. In some embodiments, a single hardware processor can be used to perform the operations of the processors 103, 104, and 128. Multiple components can be integrated within the same housing in some embodiments through permanent connections within the housing or in some embodiments through detachable wired connections such as through a USB port. In some embodiments light sensor 114 and light emitter 112 can be permanently integrated into a housing with the display module 100 and the user interface 124, while an ultrasound probe 102 is removably connected to the housing and other components through a wired connection such as a USB port. Accordingly, the NIR imaging system, the ultrasound imaging system, and/or the other medical imaging and data collection devices discussed herein can be implemented into a single platform (e.g., utilizing the same processor, the same display, the same user interface, the same memory, the same communication module, and/or the same file system). A housing containing the display module 100 can be mountable onto an articulating arm 125, which may or may not be slidably coupled to a vertical support member 127.

The multi-image display module 100 can be wearable. A wearable multi-image display module 100 can in some embodiments be mounted or attached to any part of a user, such as to the user's arm, chest, head, or shoulders. In some embodiments, a multi-image display module 100 can also be configure to display images as a heads up display. A multi-image heads up display module 100 can display multiple images on an at least partially transparent surface or the images can be projected directly onto the retina of the user's eye. The wearable display 100 can maintain the displayed image(s) in the field of view of the user regardless of movement of the user's head. In some embodiments a display module 100 can be any combination of one or more of the following: wearable, a heads up display module 100, or a multi-image display module 100. In some embodiments the multi-image display module 100 can be mountable onto an articulating arm 125, which may or may not be slidably coupled to a vertical support member 127.

In some embodiments, the ultrasound probe 102 is in wireless communication with the ultrasound image processor 104, and/or the light sensor 114 is in wireless communication with the light image processor 103. In some embodiments, wireless communication can occur between the ultrasound probe 102 and ultrasound image processor 104, and wired communication can occur between the light sensor 114 and the light image processor 103. In some embodiments, wireless communication can occur between the light sensor 114 and the light image processor 103, and wired communication can occur between the ultrasound probe 102 and ultrasound image processor 104.

In some embodiments, the light sensor 114 and/or the ultrasound probe 102 can in some embodiments wirelessly communicate with one or more remote image processors configured to display the image(s) on a remote multi-image display module (e.g., in a manner similar to FIGS. 5A and/or 5B). A remote image processor can be configured to generate an identical image or images of imaging target 106 as displayed on a local multi-image display module 100. In some embodiments a remote image processor can generate an image or images meeting different parameters from the image or images displayed on a local multi-image display module 100.

In some embodiments, an ultrasound image processor 104 and/or a light image processor 103 are in wireless communication with a multi-image display module 100. In some embodiments, only one of ultrasound image processor 104 or light image processor 103 are in wireless communication with the multi-image display module 100. Ultrasound image processor 104 and light image processor 104 can wirelessly communicate with a remote multi-image display module (e.g., similar to FIGS. 5A and/or 5B). In some embodiments a remote multi-image display module can display identical images as a local multi-image display module 100. A remote multi-image display module can be configured to display a more or less detailed set of images compared to a local multi-image display module 100.

Although pictured as separate processors in FIG. 6A, ultrasound image processor 104 and light image processor 103 can be either partially or entirely the same processor, in some embodiments. A single processor can act as both the ultrasound image processor 104 and the light image processor 103. Ultrasound image processor 104 and light image processor 103 can be separate hardware processors, in some embodiments. In some embodiments, two separate communication modules or a single communication module can facilitate the wireless communication of the ultrasound image processor 103 and the light image processor 103. In some embodiments, additional communication modules may be utilized for wireless communications. In some embodiments at least one communication module can be integrated into any of the following: the ultrasound probe 102, the light sensor 114, the ultrasound image processor 104, the light reflection image processor 103, or the multi-image display module 100. Similarly, the additional sensor processor 128 pictured in FIG. 6B can in some embodiments be incorporated partially or entirely in the same processor as ultrasound image processor 104, light image processor 103, or both. Wireless communication with an additional sensor processor 128 and/or sensor 126 can be facilitated by one or more communication modules.

In some embodiments, a user can select what image or images to display on a multi-image display module 100 through user interface 124. In some embodiments a user can direct multi-image display module 100 to display a light image (e.g., an NIR image), an ultrasound image, or both. The user interface can provide a user input elements for selecting to view both images at once on the same display, for selecting to view the ultrasound image, and/or for selecting to view the light image (e.g., the NIR image). User interface 124 can provide a user with additional options such as altering the size or position of the image or images. A user interface 124 can be utilized in combination with any other discussed image display module 100, including: a wearable image display module such as shown in FIGS. 2A, 2B, or 2C; a heads up image display module 100; or a remote image display module 100. In some embodiments, wireless communication can be utilized between user interface 124 and any other component. The user interface 124 can include buttons, switches, a touchscreen, or various other types of user input elements. In some cases the display 100 can include the user interface 124.

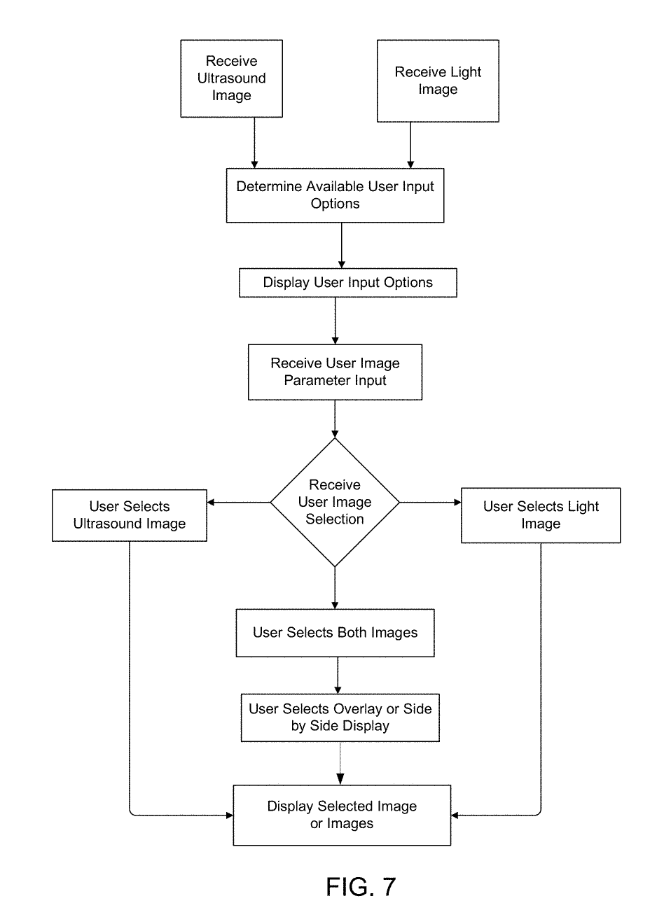

FIG. 7 shows a flowchart of an example for how a multi-image display module 100 can display an image or images in accordance with user instructions (e.g., received by user interface 124). The multi-image display module 100, or a controller configured to send one or more images to the display module 100, can receive an ultrasound image, a light reflection image, or both. The, multi-image display module 100, or a controller, can determine what user input options are available and display the available user input options. A user can supply image parameter input and/or an image selection (e.g., to the user interface 124 or to the multi-image display module 100). Multi-image display module 100 can display the selected image or images in conformance with the image parameter input. If the user selects to display both images, then user can in some embodiments select whether to display the images side-by-side or overlaid. After displaying the selected image or images, the process can be continued so that a user can input new parameters or selections.

The order of the steps shown in FIG. 7 can be changed. For example, in some embodiments, either determining what user input options are available, or receiving user image selections can be performed before receiving the ultrasound image or the light reflection image. Also, some or all of the steps shown in FIG. 7 can in some embodiments be performed by user interface 124 or any other processor instead of by multi-image display module 100. In addition to the other steps show, one or more separate steps of communicating instructions to an image processor, the light sensor, the light emitter, or the ultrasound probe may also be performed. In some embodiments, portions of the method discussed in connection with FIG. 7 can be omitted. For example, in some embodiments, the user interface can be static and the determination of the available user input options can be omitted. In some embodiments, the user interface can enable the user to select one or more images but does not enable the user to provide input relating to image parameters. Although the flowchart shown in FIG. 7 illustrates the user selecting between the light image (NIR), the ultrasound image, or both, the user interface can similarly enable the user to toggle between or select various other images and/or data for display.

Information relating to attempts to locate a vein using NIR light can be recorded by the imaging system automatically or in response to a user command received by the user interface 124. For Example, the system can be configured to store NIR images (e.g., on non-transitory computer-readable storage, in a patient file, in a hospital information system (HIS), electronic medical record (EMR), or electronic health record (HER)), as discussed, for example, in the '604 Application. For example, the system can include a communication system that can communicate with a database (e.g., on the HIS) for storing NIR images and associations between the NIR images and the associated patients and medical procedures.

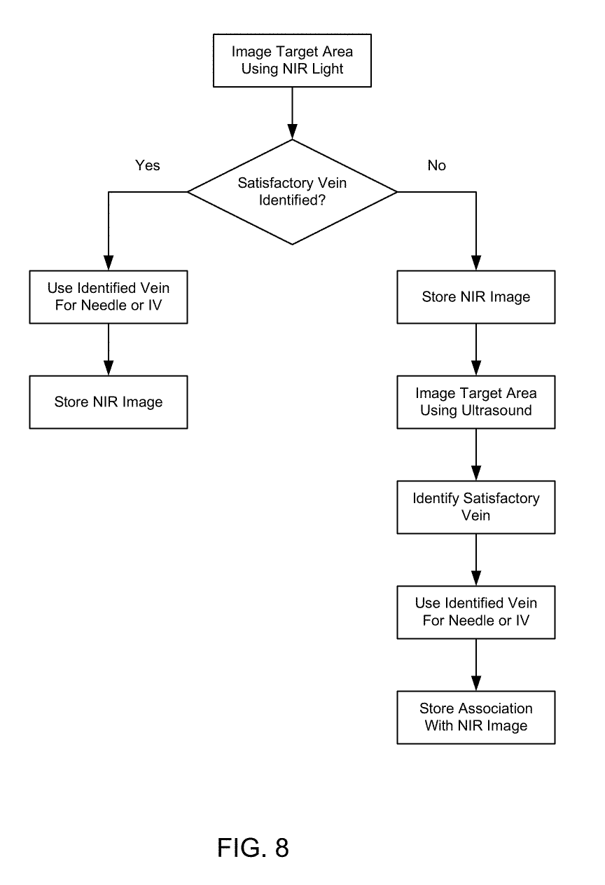

FIG. 8 shows a flowchart of an example of a process for performing and recording attempts to locate a vein or venous access site using NIR imaging and ultrasound imaging. A medical practitioner can image the target area using NIR light, such as disclosed herein and/or in the '604 Application. For example, in some embodiments, multispectral NIR imaging can be used (e.g., using a plurality of NIR light sources having different NIR emission spectra) to image the target area. If a satisfactory vein is identified using the NIR imaging system, the medical practitioner can use the identified vein to access the patient's vasculature (e.g., to insert a needle, IV, or catheter). In some embodiments, the system can store one or more images from the NIR imaging system. For example, the medical practitioner can provide a user command (e.g., via the user interface 124) to capture an NIR image of the identified vein before and/or after accessing the vein (e.g., with the needle, IV, or catheter). The system can document the condition of the identified vein before and/or after being accessed, which can facilitate evaluation and training of medical practitioners and can provide evidence that can facilitate defense of a claim of medical misconduct or mistake. If a satisfactory vein is not identified using the NIR imaging system, the system can store an indication that an attempt was made to identify a suitable vein using NIR imaging. For example, the system can store an NIR image, which can be indicative of the medical practitioner's “best effort” to identify a suitable vein using NIR imaging. The medical practitioner can provide a command (e.g., via the user interface 124) to store an NIR image that shows the target area and does not show a vein or a venous access site sufficient for the needle, IV, catheter, etc. In some cases, multiple images can be stored. For example, the system can store multiple images (e.g., a video) that document the medical practitioner's attempts to use NIR imaging to identify a satisfactory vein. The NIR image(s) can be stored in local or remote non-transitory computer-readable storage, in a patient file, in a hospital information system (HIS)), etc. such as described in the '604 Application. In some embodiments, the system can store meta data with the NIR image, and the meta data can include information regarding the settings of the system during the imaging attempt, the date and time of the imaging attempt, a patient identifier, a medical practitioner identifier, etc. The system can enable the user to add notes (e.g., via the user interface 124) that are associated with the imaging attempt and/or the stored NIR image(s). If NIR imaging is not successful, the medical practitioner can transition to using ultrasound imaging to identify a suitable vein. Ultrasound imaging can be more costly than NIR imaging and can require patient contact (e.g., using the ultrasound probe), whereas the NIR imaging can be performed without patient contact. Once a suitable venous access site is identified using ultrasound imaging, the medical practitioner can use the identified vein to access the patient's vasculature (e.g., using a needle, IV, catheter, etc.). The system can store an association between the NIR image and the patient or the ultrasound procedure. In some embodiments, the documentation can be performed automatically instead of in response to a user command.

The process of FIG. 8 can in some embodiments be useful for medical professionals to demonstrate an attempt to use NIR imaging to locate a vein before resorting to ultrasound imaging. An insurance company may be less likely to cover the more expensive ultrasound imaging costs if there is no record of previous attempts to locate a vein using less expensive methods (e.g., NIR imaging). In some embodiments, the ultrasound probe may not activate unless a record of an attempt to use NIR imaging has been recorded. In some embodiments, the system will automatically activate or prompt a user to activate the ultrasound probe in response to a detected failure to locate a vein with NIR light. The process of FIG. 8 can in some embodiments be performed automatically with no additional input from a user, other than the activation of the NIR light. In some embodiments, not all steps of FIG. 8 need to be performed, some steps can be omitted, some steps can be combined or divided into multiple steps, the order of some steps can be changed, and additional steps can be added. For example, in some embodiments, the storing of the indication of the attempt to use NIR imaging to identify a vein (e.g., storing an NIR image) can be omitted. The medical practitioner can attempt to use NIR imaging to identify a suitable venous access site and, if not successful, the medical practitioner can use ultrasound imaging (e.g., on the same display used for the NIR imaging) to identify a suitable venous access site. In some embodiments, records of all attempts to locate a vein with NIR imaging can be recorded. In some cases, the NIR imaging can be used to image a larger portion of target area than the ultrasound imaging. In some embodiments, the medical practitioner can use NIR imaging to identify a specific portion of the target area to use for the ultrasound imaging. For example, NIR imaging can be used to locate one or more veins, and the ultrasound imaging can be used to perform a more detailed analysis of the one or more veins (e.g., to determine the condition of the one or more veins and/or to confirm that the vein is suitable for accessing the patient's vasculature). The system can be used to document various components of imaging target 106, such as the patient's vasculature, the blood flow, the blood flow rate, IV patency, infiltration and/or extravasation, and/or areas that should be avoided when placing an IV (e.g., the valves and branches in the vasculature and scar tissue).

In some embodiments, the near infrared image and the ultrasound image can be displayed on the same display, as described herein. A medical imaging instrument that includes both a near infrared imaging system and an ultrasound imaging system can be used. The same processor(s) can be used to produce the near infrared image and the ultrasound image.

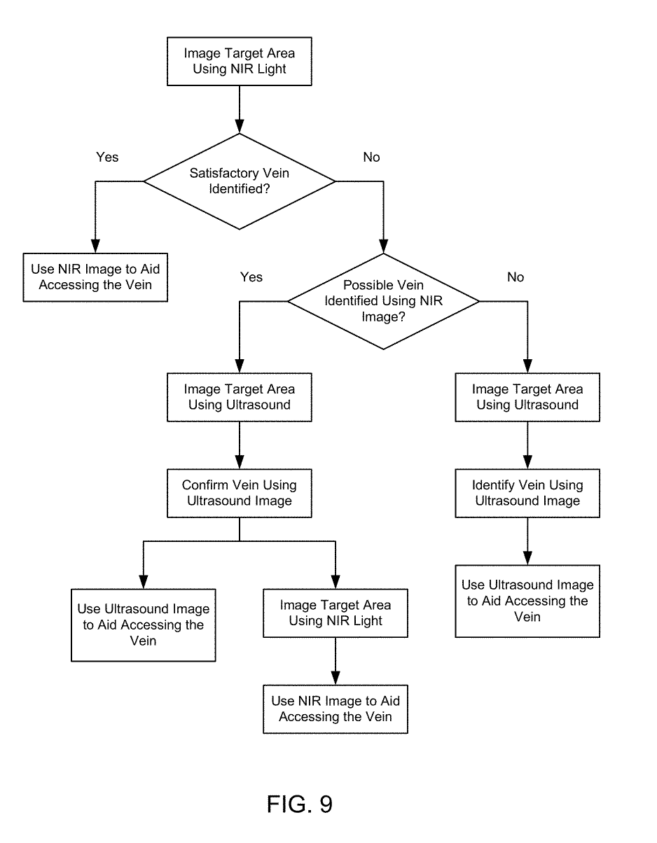

FIG. 9 shows a flowchart of an example embodiment of a method for accessing a patient's vasculature. A medical practitioner can use an NIR imaging system to image a target area on a patient. For example, the medical practitioner can illuminate the target area with near infrared light (e.g., as described herein) and can position a light sensor to receive near infrared light from the target area (e.g., reflected or scattered from the target area). The medical practitioner can position the light sensor at a distance from the target area, and the distance can at least about 1 inch, at least about 5 inches, at least about 7 inches, at least about 9 inches, at least about 10 inches, at least about 15 inches, etc. The distance can be less than or equal to about 30 inches, less than or equal to about 20 inches, less than or equal to about 15 inches, less than or equal to about 12 inches, less than or equal to about 10 inches, less than or equal to about 7 inches, less than or equal to about 5 inches, etc. If a satisfactory venous access site is identified using the NIR imaging, the medical practitioner can proceed to access the patient's vasculature using the identified vein (e.g., at the venous access site). For example, the medical practitioner can insert a needle or other medical implement into the vein at the venous access site.

If the medical practitioner was able to identify a possible venous access site using the NIR imaging, the medical practitioner can use ultrasound imaging to confirm the venous access site. For example, in some instances, the medical practitioner may see an object in the NIR image that might or might not be a vein, or the NIR image might show a vein but without sufficient image quality for the medical practitioner to determine whether the vein presents a suitable venous access site. The medical practitioner can then use an ultrasound probe to emit ultrasound signals into the target area. Echo signals can be received by the ultrasound probe and an ultrasound image can be displayed on the display, as discussed herein. The same display can be used for displaying the NIR images and the ultrasound images. The medical practitioner can use the ultrasound image to confirm the presence of a vein (e.g., that presents a suitable venous access site). Once the vein is confirmed using ultrasound imaging, the medical practitioner can use the vein to access the patient's vasculature. For example, the medical practitioner can insert a needle or other medical implement into the identified vein (e.g., at the identified venous access site). The medical practitioner can use ultrasound imaging and/or NIR imaging to facilitate inserting the needle or other medical implement, for example, depending on the preference of the medical practitioner. By way of example, the medical practitioner might see an object (e.g., a dark area) in the NIR image that could possibly be a vein. The medical practitioner can use ultrasound imaging to image the identified object to confirm that the object is indeed a vein. Then the medical practitioner can revert back to NIR imaging and can use the NIR image of the object, which was confirmed to be a vein, to facilitate insertion of a needle into the vein.

If the NIR image is not sufficient to identify a possible vein or venous access site, the medical practitioner can use ultrasound imaging to image the target area and to identify a suitable venous access site. The medical practitioner can then use the ultrasound imaging to facilitate insertion of the medical implement into the identified vein (e.g., at the venous access site).

In some embodiments, the system can be configured to automatically evaluate the NIR. If the evaluation of the NIR image determines that the NIR image is insufficient for identifying a suitable vein or vascular access site, the system can output an alert (e.g., a sound or a visual alert on the display 100). For example, the alert can be an instruction to use ultrasound imaging. In some embodiments, the system can use image contrast analysis and/or edge detection algorithms to evaluate the NIR image.

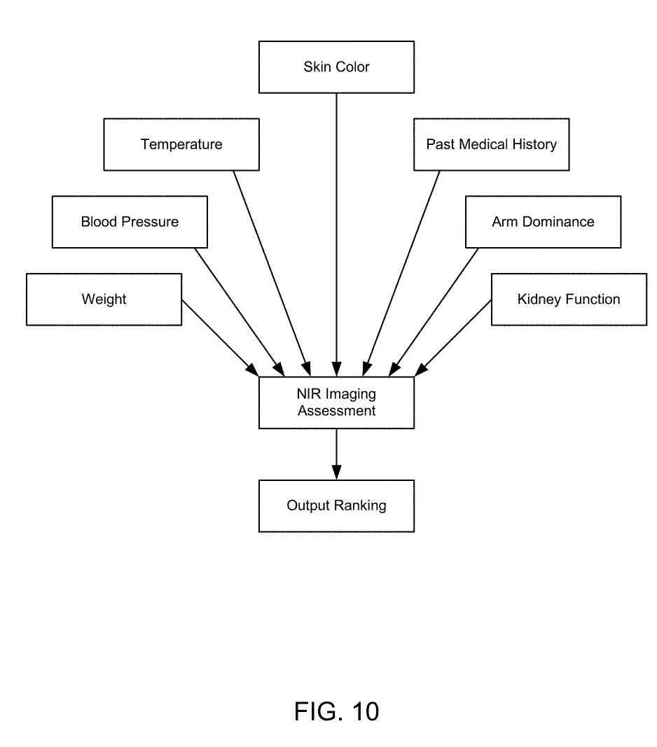

With reference to FIG. 10, in some embodiments, a system (e.g., the medical imaging systems disclosed herein) can be configured to perform an assessment of the likelihood that NIR imaging would be successful for identifying a suitable vein to provide a venous access site. A processor can be used to consider one or more factors to perform the NIR imaging assessment. The processor can be an application specific integrated circuit (ASIC), or a general purpose processor configured to execute instructions stored on computer-readable memory to implement the assessment. The system can use a formula, algorithm, or look-up-table to generate a score (e.g., a ranking value) indicative of how likely it is that NIR imaging will be able to identify a suitable venous access site. Various factors can be considered by the assessment, including one or more of the following: the patients weight, Body Mass Index (BMI), blood pressure, temperature, skin color, past medical history (e.g., received from a patient file or hospital information system, such as using a wireless or wired connection), arm dominance (right or left handed), kidney function, etc. The system can include a user interface (e.g., user interface 124) that is configured to enable a medical practitioner to input patient information to be used in the assessment. For example, the user interface can enable input of the patient's weight, BMI, blood pressure, temperature, skin color, medical history, kidney function, etc. In some embodiments, the system can be configured to measure one or more of the factors to be used by the assessment. For example, the system can include one or more medical devices, or one or more communication modules (e.g., electrical ports or wireless communication modules) configured to receive data from one or more medical devices, that are configured to measure one or more of the factors. For example, a scale, blood pressure monitor, a thermometer, a spectrometer, etc. can provide data to the system to be used in making the NIR imaging assessment. In some embodiments, the system can provide a single platform for performing NIR imaging and for measuring one or more factors for use in assessing the likelihood of success of NIR imaging for facilitating vascular access.

The system can output (e.g., using the user interface 124) information relating to the assessment. For example the system can output (e.g., as an image on the display 100) a raking indicating how likely it is that NIR imaging will be able to identify a suitable venous access site. For example, a Likert-type scale can be used with provide a ranking between 1 to 5 (e.g., with 1 indicating that a vein is likely to be easily identifiable using NIR imaging and with 5 indicating that a vein is very unlikely to be identifiable using NIR imaging). A medical practitioner can use the system to decide whether to skip NIR imaging, for example, and use ultrasound imaging to identify a vein. The system can be configured to document the NIR imaging assessment. For example, information regarding the assessment (e.g., the ranking and/or the factors using making the assessment) can be stored in local or remote non-transitory computer readable storage such as a patient file or hospital information system. A association can be stored between the assessment information and the patient and/or the ultrasound procedure. The documentation can be useful to demonstrate (e.g., to an insurance company) why ultrasound imaging was used instead of NIR imaging to facilitate venous access.

The systems and methods disclosed herein can be implemented in hardware, software, firmware, or a combination thereof. Software can include computer-readable instructions stored in memory (e.g., non-transitory, tangible memory, such as solid state memory (e.g., ROM, EEPROM, FLASH, RAM), optical memory (e.g., a CD, DVD, Blu-ray disc, etc.), magnetic memory (e.g., a hard disc drive), etc.), configured to implement the algorithms on a general purpose computer, special purpose processors, or combinations thereof. For example, one or more computing devices, such as a processor, may execute program instructions stored in computer readable memory to carry out processes disclosed herein. Hardware may include state machines, one or more general purpose computers, and/or one or more special purpose processors. In some embodiment, multiple processors can be used, and in some implementations the processors can be at different locations (e.g., coupled via a network). While certain types of user interfaces and controls are described herein for illustrative purposes, other types of user interfaces and controls may be used.

The embodiments discussed herein are provided by way of example, and various modifications can be made to the embodiments described herein. Certain features that are described in this disclosure in the context of separate embodiments can also be implemented in combination in a single embodiment. Conversely, various features that are described in the context of a single embodiment can be implemented in multiple embodiments separately or in various suitable subcombinations. Also, features described in connection with one combination can be excised from that combination and can be combined with other features in various combinations and subcombinations. Various features can be added to the example embodiments disclosed herein. Also, various features can be omitted from the example embodiments disclosed herein.

Similarly, while operations are depicted in the drawings or described in a particular order, the operations can be performed in a different order than shown or described. Other operations not depicted can be incorporated before, after, or simultaneously with the operations shown or described. In certain circumstances, parallel processing or multitasking can be used. Also, in some cases, the operations shown or discussed can be omitted or recombined to form various combinations and subcombinations.

1. A method for displaying an ultrasound image, the method comprising:
identifying a target body portion of a patient to be imaged;
emitting ultrasound signals from an ultrasound probe, wherein the ultrasound signals cause echo signals to be reflected from tissue within the target body portion;
receiving the echo signals;
generating an ultrasound image of the target body portion based at least in part on the echo signals; and
displaying the ultrasound image on a head-mounted display.

2. The method of claim 1, further comprising:
transmitting the ultrasound image to a remote display accessible to a remote healthcare professional;
receiving treatment instructions from the remote healthcare professional; and
administering treatment based at least in part on the instructions from the remote healthcare professional.

3. The method of claim 1, further comprising:
generating echo signal data based at least in part on the echo signals;
transmitting the echo signal data wirelessly to an image processor that generates the ultrasound image.

4. The method of claim 1, further comprising transmitting the ultrasound image wirelessly to the head-mounted display.

5. The method of claim 1, further comprising attaching the head-mounted display to a user.

6. The method of claim 5, wherein the attaching is achieved by the user wearing a helmet that supports the head-mounted display such that the ultrasound image displayed on the head-mounted display is configured to be visible to the user wearing the helmet.

7. The method of claim 5, wherein the attaching is achieved by the user wearing an eyewear frame that supports the head-mounted display such that the ultrasound image displayed on the head-mounted display is configured to be visible to the user wearing the eyewear frame.

8. The method of claim 5, wherein the attaching is achieved by the user wearing a headband that supports the head-mounted display such that the ultrasound image displayed on the head-mounted display is configured to be visible to the user wearing the headband.

9. A system for displaying an ultrasound image, the system comprising:
an ultrasound probe configured to emit ultrasound signals and detect echo signals reflected from tissue in a target body portion of a patient;
an image processor configured to receive echo signal data that is based at least in part on the echo signals received by the ultrasound probe and generate an ultrasound image of the target body portion based at least in part on the echo signal data; and
a wearable display configured to receive the ultrasound image of the target body portion from the image processor and display the ultrasound image.

10. The system of claim 9, further comprising a remote display accessible to a remote healthcare professional and configured to receive and display the image of the target body portion.

11. The system of claim 9, further comprising a communications module that is configured to deliver the ultrasound image wirelessly to a remote display accessible to a remote healthcare professional.

12. The system of claim 9, further comprising a communications module that is configured to deliver the ultrasound image wirelessly to the wearable display module.

13. The system of claim 9, wherein the wearable display is configured to display the image of the target body portion on a heads-up display.

14. The system of claim 9, wherein the wearable display comprises an at least partially transparent surface.

15. The system of claim 9, further comprising a helmet that supports the wearable display such that the ultrasound image displayed by the wearable display is configured to be visible to a person wearing the helmet.

16. The system of claim 9, further comprising an eyewear frame that supports the wearable display such that the ultrasound image displayed by the wearable display is configured to be visible to a person wearing the eyewear frame.

17. The system of claim 9, further comprising a headband that supports the wearable display such that the ultrasound image displayed by the wearable display is configured to be visible to a person wearing the headband.

18. An image display system comprising:
an ultrasound probe;
a processor configured to generate an image based at least in part on data from the ultrasound probe; and
a wearable display configured to display the image.

19. The system of claim 18, wherein the wearable display is further configured to display the image as a heads-up display image.

20. The system of claim 18, wherein the processor is capable of wireless communication with at least one of the ultrasound probe and/or the wearable display.

21.-114. (canceled)
